(19) United States
(12) Patent Application Publication
(10) Pub. No.: US 2014/0088684 A1
(43) Pub. Date: Mar. 27, 2014

(54) CATHETER SYSTEM

(71) Applicant: LARRY D. PASKAR, CHESTERFIELD, MO (US)

(72) Inventor: LARRY D. PASKAR, CHESTERFIELD, MO (US)

(21) Appl. No.: 14/093,463

(22) Filed: Nov. 30, 2013

Related U.S. Application Data
(63) Continuation of application No. 13/843,823, filed on Mar. 15, 2013.
Application No. 13/843,823 is a continuation of application No. 12/660,489, filed on Feb. 26, 2010.
Application No. 12/660,489 is a continuation of application No. 12/454,420, filed on May 18, 2009, now abandoned.
Application No. 12/454,420 is a continuation of application No. 11/434,368, filed on May 15, 2006.

(51) Int. Cl. A61F 2/95; A61M 25/01

(52) U.S. Cl. CPC A61F 2/95; A61M 25/0147
USPC 623/1.11; 604/95.05

(57) ABSTRACT

A catheter system and method that includes a catheter element sized to pass through blood vessels to an organ of an animal. A medical element can be disposed with respect to the catheter element so as to interact therewith, the medical element being capable of assuming and maintaining a curved configuration. An ablating or mapping tool can be disposed on either the medical element or the catheter element, or both. The catheter system can be used for inserting a stent graft limb branch into a stent graft body disposed in a living being, the stent graft body having a trunk and an opening for receiving said branch. A guide element is inserted through a vessel of the living being to the vicinity of the branch receiving opening of the stent graft body. The catheter system can include a hand control for manipulating the catheter system.

### CROSS-REFERENCE TO RELATED APPLICATIONS

This is a continuation of co-pending U.S. patent application Ser. No. 11/434,368 filed on May 16, 2006; U.S. patent application Ser. No. 12/454,420 filed on May 18, 2009; U.S. patent application Ser. No. 12/660,489 filed on Apr. 26, 2010; and U.S. patent application Ser. No. 13/843,823 filed on Mar. 15, 2013, and the entire contents of each of those applications are hereby incorporated by reference as if fully stated herein.

### STATEMENT REGARDING FEDERALLY SPONSORED RESEARCH OR DEVELOPMENT

Not Applicable.

### FIELD

The present disclosure relates to a catheter system adaptable for use in executing various medical procedures within an animal.

### BACKGROUND

This section provides background information related to the present disclosure which is not necessarily prior art.

A catheter is a tubular medical device for insertion into canals, vessels, passageways, or body cavities for diagnostic or therapeutic purposes such as to permit injection/withdrawal of fluids, to keep passageways open, to inspect internal organs and tissues, and to place medical tools into position for medical treatment within the body of an animal.

There is a wide range of medical procedures where a catheter may be used. For example, a catheter may be used to assist the elimination of urine by insertion of a urethral catheter into a patient's body. In other applications, a catheter may be used for treatment of abdominal aortic aneurysm, clearing of obstructions within a carotid artery, placing a pacing wire in the coronary veins of a heart, placement of a feeding tube or other nasogastric or nasoenteric tubes in a living being, or for colonoscopies.

An abdominal aortic aneurysm is a weakening, or balloon dilatation, of the major artery of the body, the aorta, often in the abdomen below the level of the renal arteries. This weakening of the wall of the aorta may be secondary to degenerative arteriosclerosis or predisposing genetic conditions, infection, inflammation or trauma. When the size of the aneurysm reaches a critical value of 5 cm in diameter, there is a real threat of aneurysm leak or rupture and a true medical emergency which can result in death due to hemorrhage.

Open abdominal surgical repair with placement or reinforcement of the dilated weakened segment of the aorta, using an artificial replacement graft segment, has long been practiced but is a difficult procedure for the patient with significant morbidity and mortality. In recent years, innovative minimally invasive surgical procedures have been developed. Rather than cutting through large areas of healthy tissue (such as making large incisions in the abdomen for gallbladder surgery for example), several small holes are made to allow passage of instruments through which the operative site can be viewed and the tissue, organ, etc. removed and/or treated. This spares the patient the major trauma of a large, slow healing open wound with attendant secondary complications related to slow recovery time, complications of longer surgery with its attendant risks. Recovery from laparoscopic cholecystectomy and other such minimally invasive procedures have proved revolutionary in medicine. Similar excellent results have recently been achieved using orifice surgery.

In keeping with the desire to develop a system for repairing an abdominal aortic aneurysm in a minimally invasive manner, stent grafts have been developed which can be delivered through a blood vessel to the aortic aneurysm, becoming a new conduit through which blood flows, so that the weakened aneurysmal segment is not exposed to high blood pressure and the attendant threat of aortic leak or rupture.

The stent graft can be delivered through the femoral artery on one side, and advanced to the weakened aneurysm, fixing the upper portion of the stent to the normal non-dilated infrarenal segment of the aorta, tracking as a new excluding artificial lumen segment within the weakened ballooned aneurysm, and continuing as branch components into the aortic bifurcation and normally branched native iliac arteries. The new co-axially placed artificial segment bridges the aorta from its normal infrarenal portion, mimicking the vascular anatomy branches into the left and right iliac arteries to isolate the weakened native aneurysmal portion from the threatening blood flow pressure by re-establishing normal flow through the channels of the artificial stent graft.

The prior art in the field provides relatively accurate self-delivery of the stent and creates strong, yet flexible stent grafts with good fixation and sealing proximally and distally of the stent graft. One step, which has not been optimized, is the method and means for easily passing a guidewire or such from the modular segment in the contralateral iliac artery into the short limb branch of the body of the stent graft, thereby allowing advancement of the modular segment tracking over various wires, or such, to ultimately telescope, seat and seal into the short limb, mimicking the native anatomy of the aorta and iliac bifurcation. An hour and-a-half procedure may become a three hour procedure while various shaped catheters, guidewires and guiding catheters are tried in an attempt to match the needed shape configuration and tip direction angulation and orientation with a complex anatomical pathway made more circuitous and serpiginous by inherent vascular dilatation and tortuosity of native vessels as well as the uncertainty of the anterior/posterior, medial/lateral relative positions of the short limb member position with respect to the modular short limb component.

What is needed and, heretofore, does not exist for this endograft stent procedure, is a medical tube that could be shaped and repeatedly reshaped, as necessary, at will, into a vast number of various curves with compound and/or complex distal end configurations which can be up-going, down-going out-of-plane as needed to allow custom formation of the appropriate distal end curve shape to match very complex anatomical and positional variations inherent in the patients undergoing these procedures.

Currently, the native anatomy and resulting segment positions cannot be known or easily anticipated prior to the procedure to allow one to chose the appropriate medical tube shape, nor can the shape and precise position of the catheter tip be finely or coarsely adjusted during the procedure to accommodate for subtle variations which would allow for expeditious crossing of the modular segments. In fact the shape of the particular medical tube which is chosen at the beginning of the procedure could result in misalignment in all three dimensions.

The ‘holy grail’ for precise navigation and negotiation of a medical tube through unknown, unpredictable, tortuous, serpiginous, body viscus, vessels, chambers, passages, spaces (potential and pathologic) is a guide system whose distal end could be configured and reconfigured ‘on the fly’ in real-time, in situ, in the body with imaging guided corrective feedback allowing coarse and fine readjustments in the shaped configurations and tip orientations with respect to its intended target as needed, to accommodate to the anatomical contour of the passage or space to be traversed. A system which would allow effective and repeated shaping and reshaping to form the precise shape necessary for the particular situation at hand would be extremely useful in this procedure.

Abnormal heart rhythms (arrhythmias) can occur as a result of accessory pathways or dual AV nodal pathways as seen in the Wolfe-Parkinson-White (W-P-W) syndrome, or as a result of electrical abnormalities in the atria or in the ventricles. Examples include atrial tachycardias, atrial flutter, atrial fibrillation, and AV node tachycardias as well as ventricular tachycardias. Many of these arrhythmias can be cured by identifying the abnormal conduction pathway within the cardiac tissue using electrophysiologic studies, i.e., mapping of cardiac activation sequences, and then disrupting or ablating the tissue containing the abnormal tissue through application of energy. The most common energy source to date is radio-frequency energy, although high voltage, direct current, laser, ultrasound, and microwave energy have also been used.

Historically, mapping and ablation were carried out through cardiac surgery which required general anesthesia and cardiopulmonary bypass. The risk was substantial, although the risk was primarily that inherent in the medical procedure and not the mapping and ablation per se. To generally reduce that risk, catheter-based techniques have been developed for mapping and ablation. Such techniques do not require major surgery and, as a result, have significantly decreased side effects and complications.

Patients with Wolfe-Parkinson-White syndrome (W-P-W) in particular are effectively and safely cured by catheter-based ablation techniques. Patients with atrial flutter and focal atrial tachycardias may selectively have their condition controlled by using catheter ablation. The W-P-W syndrome results from an anomalous conduction pathway connecting the atrial muscle directly to the ventricular muscle, thereby short circuiting and bypassing the H-P system. Destruction or ablation of this abnormal pathway can be carried out by delivering energy to the tissue containing the anomalous pathway through an electrode catheter placed in contact with the heart tissue in the left or right heart that can be reached via the peripheral arterial or venous routes commonly used for catheter procedures.

Anomalous conduction pathways can occur between the left atrium and ventricle or between the right atrium and ventricle. Mapping and ablation catheters can be placed in the left heart by one of two approaches. In the first approach, the catheters are introduced into the femoral artery and advanced into the thoracic aorta and through the aortic valve into the left ventricle. The catheter tip is positioned under the mitral valve annulus as close to the abnormal conduction pathway as possible. In the second approach, the catheters are introduced into to the left heart via the left atrium, which is entered through a trans-septal puncture in the fossa ovalis of the interatrial septum from the right atrial side. In this approach, the catheter is introduced into the right atrium through the right femoral vein and the mapping or ablation catheters are positioned on top of (and/or below) the mitral valve annulus as close to the anomalous pathway as possible via the transeptal approach.

Anomalous pathways in the right heart between the right atrium and right ventricle can be approached by one of two venous access routes. The first route targets the pathways ventricular insertion site and is carried out by entering the right atrium from either the inferior or superior vena cava, crossing the tricuspid valve into the right ventricle, and advancing the catheter to the right ventricular apex, causing it to reverse its path backwards so that the catheter tip comes to lie under the tricuspid valve region. The right ventricular insertion under the tricuspid valve is thereby mapped and ablated via this approach.

The second right heart route involves entering the right atrium from the inferior or superior vena cava and locating the atrial insertion site of the anomalous accessory pathway on top of the tricuspid valve annulus on the atrial aspect of the tricuspid valve and then ablating the atrial insertion site.

Successful entry into the heart chamber of interest in no way assures that the tip of a mapping or ablation catheter can precisely be positioned at the focus of an anomalous pathway. It is even less sure that the catheter position can be substantially fixed in a long-term stable position for the ablation procedure, given the cardiac motion as well as the marked variability in chamber size, shape, and internal structural anatomy. Steerable catheters have partially solved this problem of precise positioning and fixation by allowing variable tip curvature radii, but they remain limited in the tip curvatures possible. These steerable catheters do not permit the compound, complex, and out-of-plane shaping capabilities which may be necessary to reach the endocardiac focus by allowing custom curvature of the catheter to conform to the inner contour of the cardiac chamber, thereby permitting wedging of the catheter and subsequent fixation of its tip on the focus of interest.

In past attempts to resolve these issues many different fixed shapes that are disposed at the distal end of a catheter have been used. For example, in catheterization of the left carotid artery of a patient, the end of the catheter can be a very simple shape and still be acceptable for routing the catheter through the patient's body. In a young patient, a simple shape may also be used because the course of the vessels and the shape and size of the vessels and heart are fairly straightforward. As a patient ages, however, the arteries often become dilated and tortuous, resulting in branch vessels coming off at angles different than in the young patient. Likewise, an aged heart may become dilated and deformed, and beat in an irregular rhythm, requiring the catheter to be stabilized against the opposite wall in the heart or a vessel during the routing or treatment procedure. In such circumstances, a single, particular shape might work to route the catheter through the older patient's body, but that specific shape is not determinable until the routing process has already begun. And, if you choose the wrong shape initially when routing the catheter through the older patient, the catheter must be removed and the shape initially selected must be exchanged for a different curved catheter. That process might need to be repeated several times until the selected curve eventually accomplishes the routing of the catheter through that particular area of the patient's body. This can be particularly problematic during heart mapping and ablation procedures and can often take many hours to complete, and in fact, some procedures are ultimately unsuccessful due to the difficulty in routing the catheter precisely to reach and fix upon the focus of aberrant activity. These same routing issues occur during medical treatments that require endoscopes, gastroscopes, laparoscopes, ET tubes, nasogastric tubes, or anywhere a tube must be precisely guided and stabilized from afar.

Other previous inventions use a single pullwire to generate a curve or flatten the distal end of a catheter. However, generating a single curve rarely resolves the complex routing issues that occur during the routing of a catheter through the complex three dimensional tissues and organs of a patient's body. Those types of problematic routing issues require two curves that can be shaped, in situ, and that can be rotationally oriented as needed to pass the catheter system through the patient's body.

Thus, it is clear that a very significant problem exists and that there is a need for a catheter system having a means to shape the distal end of a medical catheter tube in situ, in real time, and under fluoroscopic guidance to modify the shape as necessary to conform to the shape of the vessels or heart chamber, for instance. It is also clear that the catheter system needed must be able to be shaped by hand controls to manipulate the distal end of the catheter, and any elements attached thereto, into either an up-going, down-going, or out of-plane configuration, and must be able to do so whether tissue can be used as a footing for the catheter to push against for forming those configurations, or whether the configurations needs to be generated in a three dimensional space completely free from any surface that might act as a footing for the catheter. Many of these same issues exist in using a catheter system for placement of pacing wires in the coronary veins of a heart, placement of feeding tubes or other nasogastric or nasoenteric tubes in a living being, and for colonoscopies. Such a shapeable catheter system would obviate exchanges of catheter components while decreasing vessel injury, patient exposure, and other associated morbidity.

### BRIEF SUMMARY OF THE INVENTION

This section provides a general summary of the disclosure, and is not a comprehensive disclosure of its full scope or all of its features.

In accordance with the various embodiments of the present invention, this invention relates to a basic catheter system having improved placement and fixation capabilities that can reduce the amount of time required in performing medical procedures. Depending on the intended use of the basic catheter system, a medical device or tool can be operatively connected to the distal end of the inner element of the basic catheter system to convert the basic catheter system into a specifically embodied catheter system to perform a specific type of medical procedure related to such instances as for example, placement of pacing wires in the coronary veins of a heart, placement of feeding tubes or other nasogastric or nasoenteric tubes in a living being, and for colonoscopies.

Embodiments of the present catheter system can be shaped by hand controls to manipulate the distal end of the catheter, and any elements attached thereto, into either an up-going, down-going, or out of-plane configuration, and can do so whether those configurations need to be generated in a three dimensional space completely free from any surface that might act as a footing for the catheter, or whether tissue is present that can be used as a footing for the catheter to push against for mechanical assistance in forming those configurations. The shaping of the catheter system is accomplished solely by manipulation of a catheter manipulator operatively connected to the catheter system, but which is located outside the physical body of the patient.

Certain embodiments of the invention provide a catheter system that allows for the interaction of a shapeable distal curve on an inner element of the catheter with a shapeable distal curve on an outer element of the catheter by rotating the inner and outer elements with respect to each other. Some embodiments of the invention include coaxial inner and outer elements that allow a pullwire to shape and generate the curve on the outer element. In those embodiments, the inner element may have a fixed shape that is positioned through activations of the pullwire or weaknesses on the outer element. In alternative embodiments, an additional pullwire may be applied to shape and generate the curve on the inner element. The catheter manipulator can be used to rotate the distal curves on one or both of the inner and outer elements to result in a wide range of shapes to form whole families of down-going, up-going and out-of-plane shapes that can be formed to generate almost any shaped, complex, compound curve in the catheter system. Certain other embodiments of the present invention are used for placement of pacing wires in the coronary veins of a heart, placement of feeding tubes or other nasogastric or nasoenteric tubes in a living being, and for colonoscopies.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

In the accompanying drawings which form part of this specification:

FIGS. 1A-A through 1B-A, and 1D-A are side elevations illustrating the construction of the catheter system of the present invention;

FIG. 1C-A is a top plan of the proximal portion of the catheter system of the present invention;

FIG. 1E-A is a view, similar to FIG. 1D-A but on an enlarged scale, illustrating the construction of a side port of the catheter system of the present invention;

FIGS. 2A-A through 2D-A are simplified elevations illustrating some of the myriad shapes which are attainable with the catheter system of the present invention; and

FIGS. 3A-A through 3C-A are simplified elevations illustrating the reformation of a complex catheter shape inside the human body.

FIGS. 4A-B through 4D-B are partial elevations illustrating operation of one embodiment of the present invention;

FIG. 5-B is a partial elevation illustrating a gap shape different from that shown in FIGS. 4A-B through 4D-B;

FIG. 6-B is a view similar to FIG. 5-B illustrating another gap shape;

FIG. 7-B is a view similar to FIG. 4A-B illustrating an alternative construction;

FIG. 8-B is a view similar to FIG. 4A-B showing a covering over the gaps;

FIG. 9-B is a sectional view illustrating an alternative way to cause the catheter to bend in a desired direction;

FIGS. 10-B and 11-B illustrate the method of construction of an alternative to that of FIGS. 4A-B through 4D-B; and

FIGS. 12-B and 12A-B are perspective views illustrating an improved handle of the present invention.

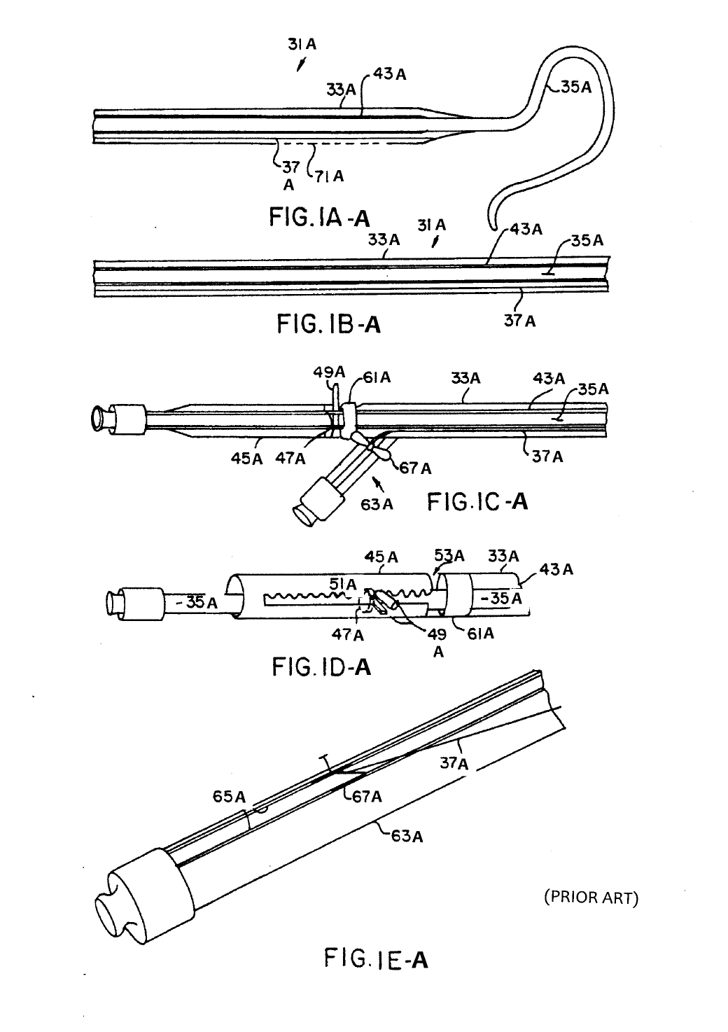

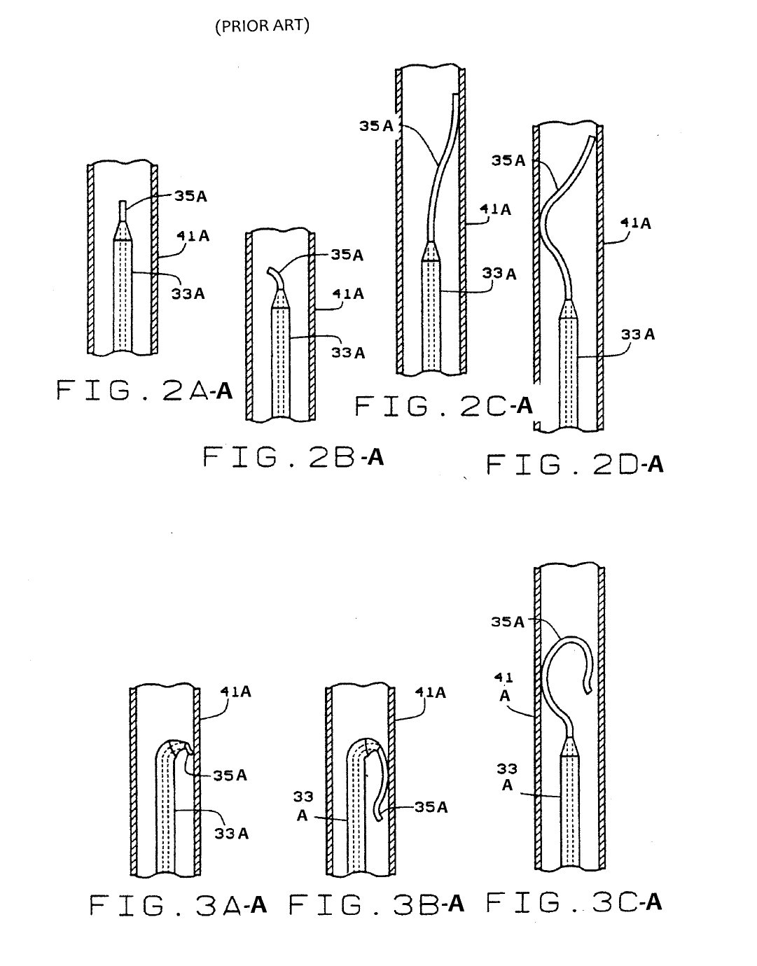

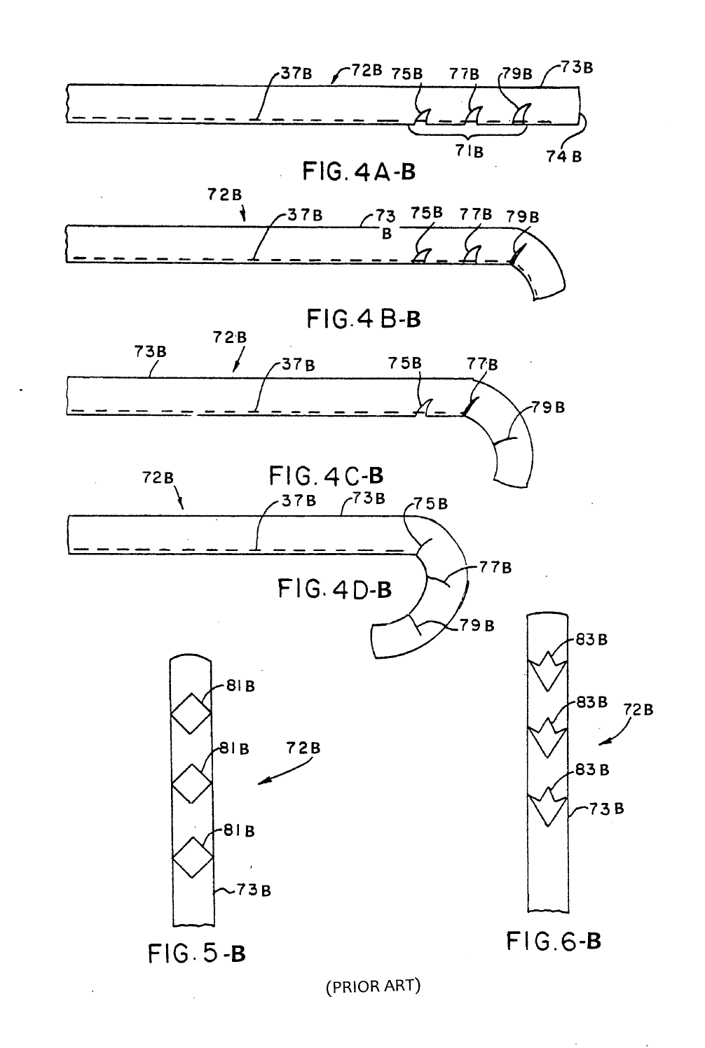

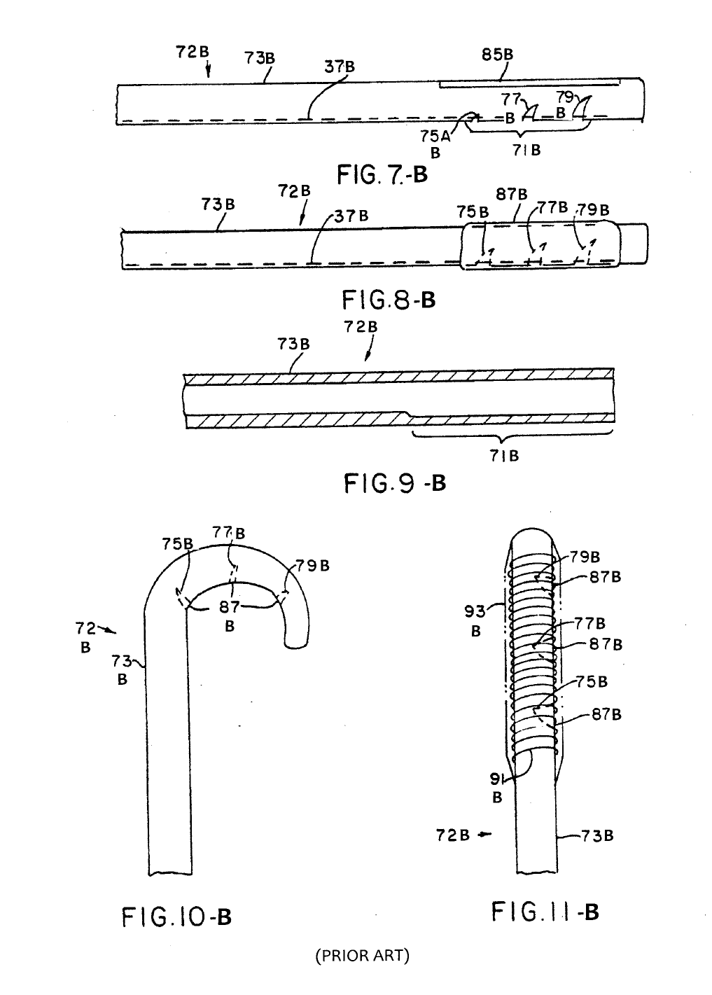

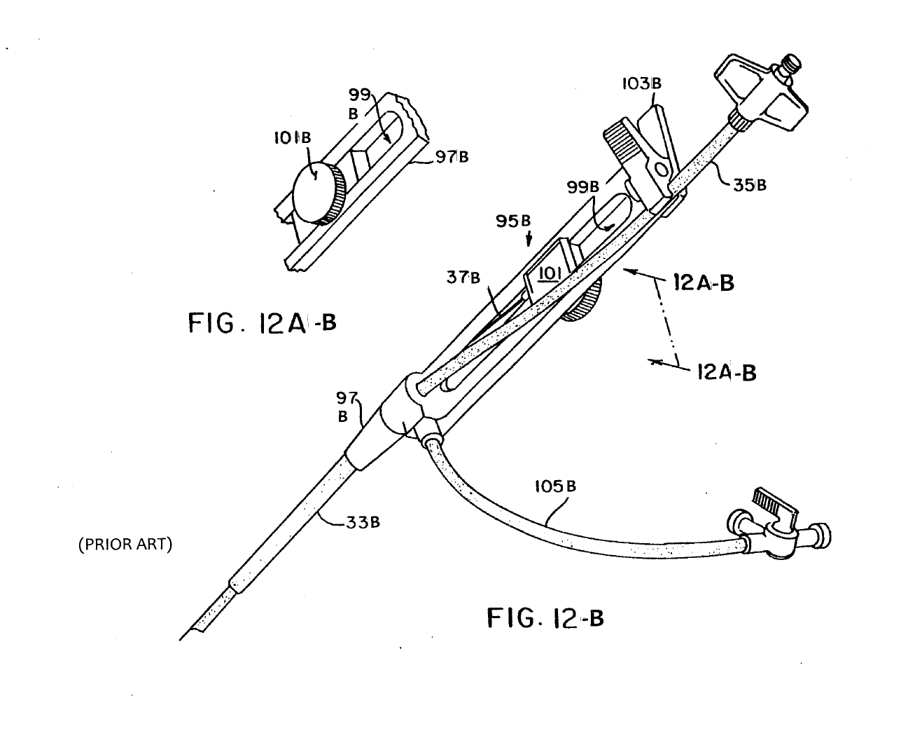

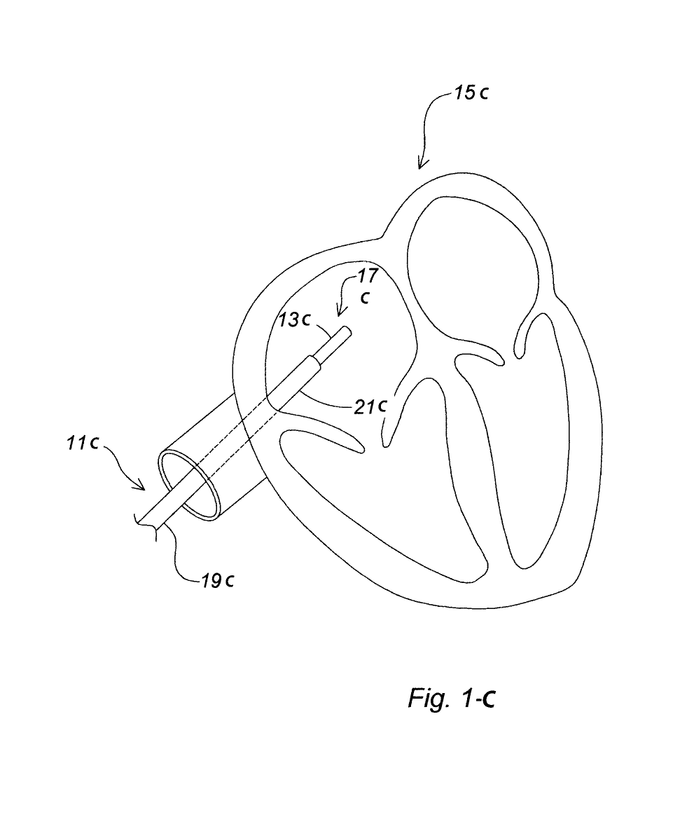

FIG. 1-C is a cross-sectional view of one embodiment of the present invention showing a catheter system being inserted in a heart;

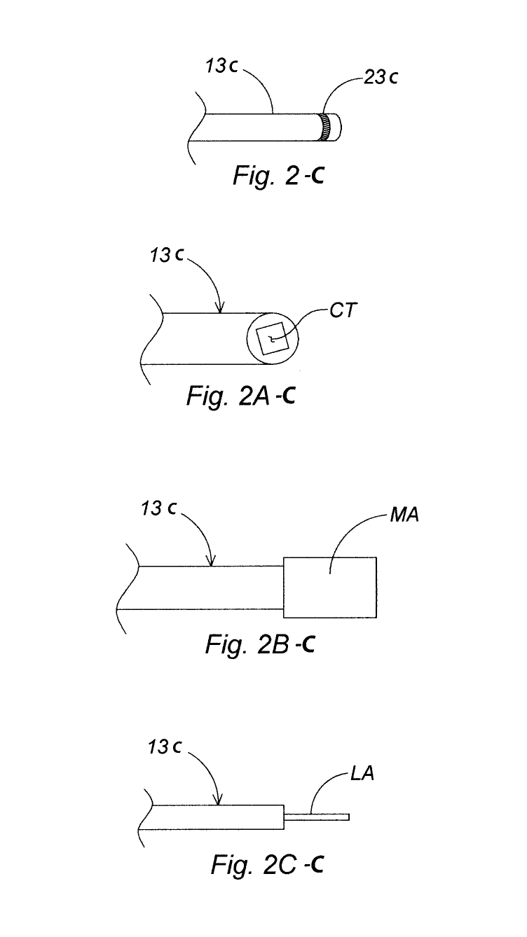

FIG. 2-C is a front elevation of the inner medical element of one embodiment of the catheter system showing a possible location of a mapping or ablating electrode;

FIG. 2A-C is a view similar to FIG. 2-C, but shown with a cryo-surgery ablation tool;

FIG. 2B-C is a view similar to FIG. 2-C, but shown with a microwave ablation tool;

FIG. 2C-C is a view similar to FIG. 2-C, but shown with a laser ablation tool;

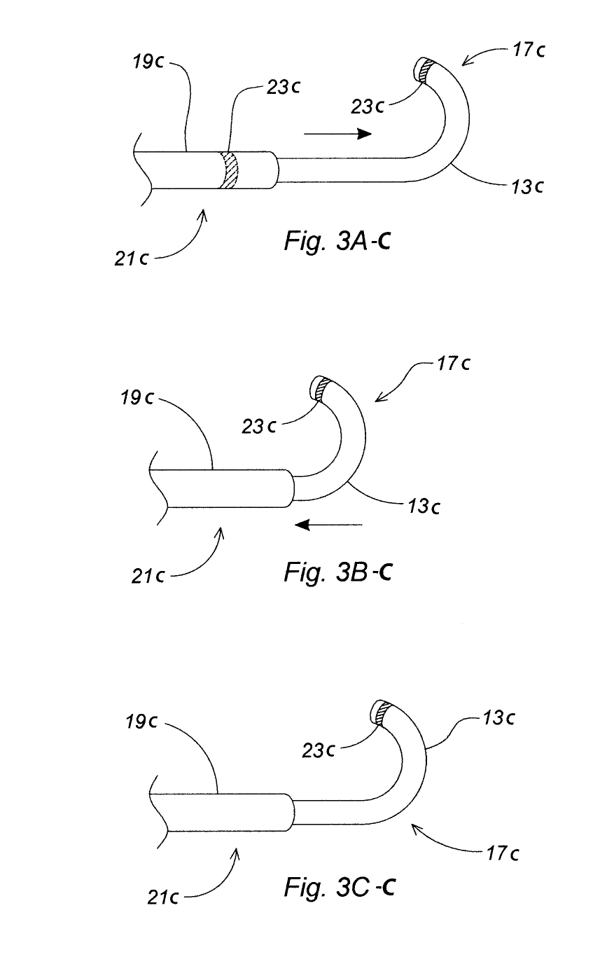

FIG. 3A-C through 3C-C are diagrams illustrating exemplary shapes made by moving the medical element translationally with respect to the catheter in which it is located for one embodiment of the present invention;

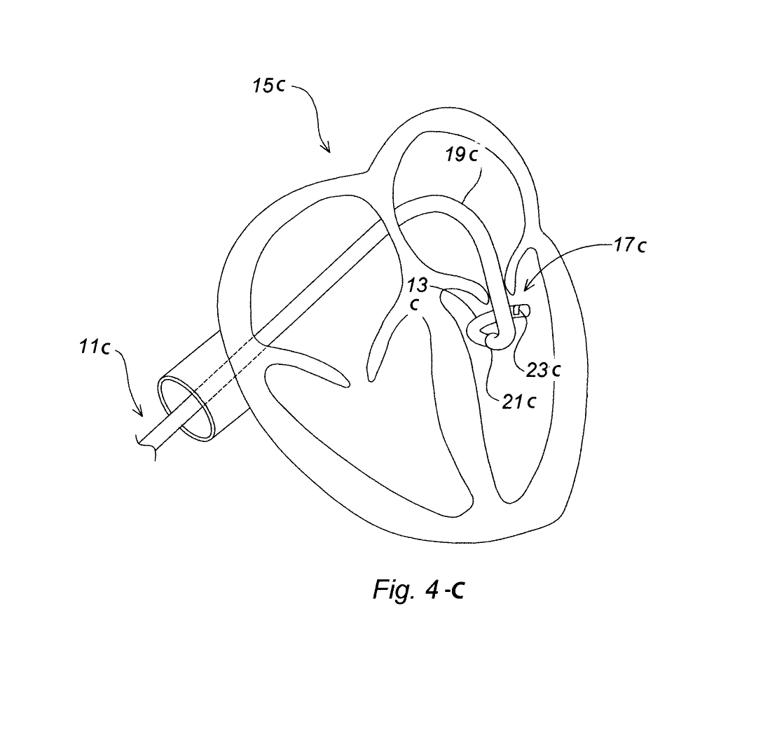

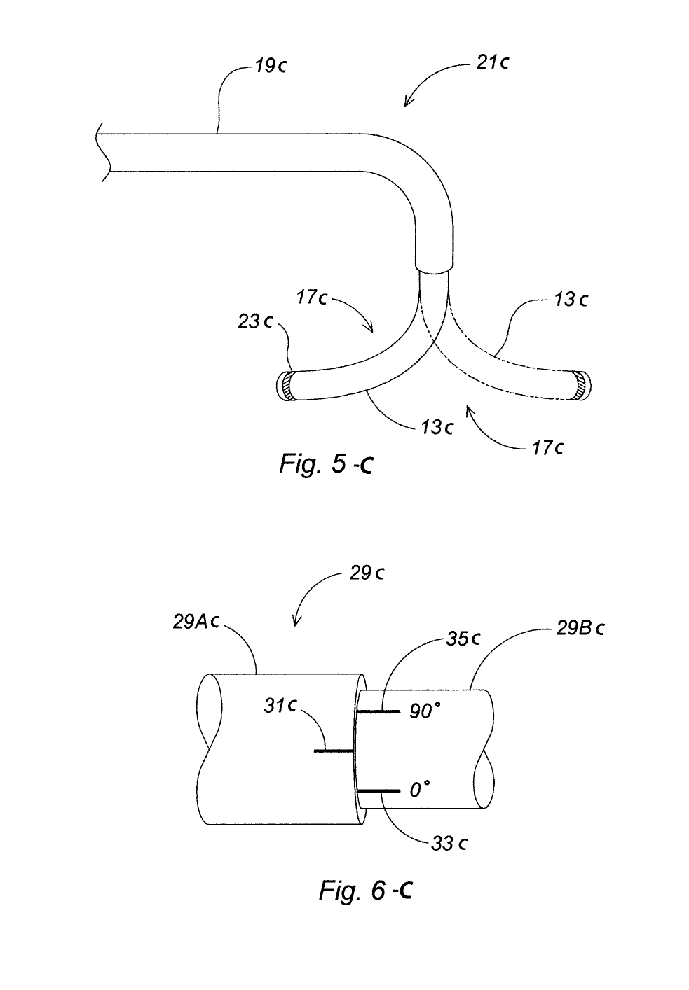

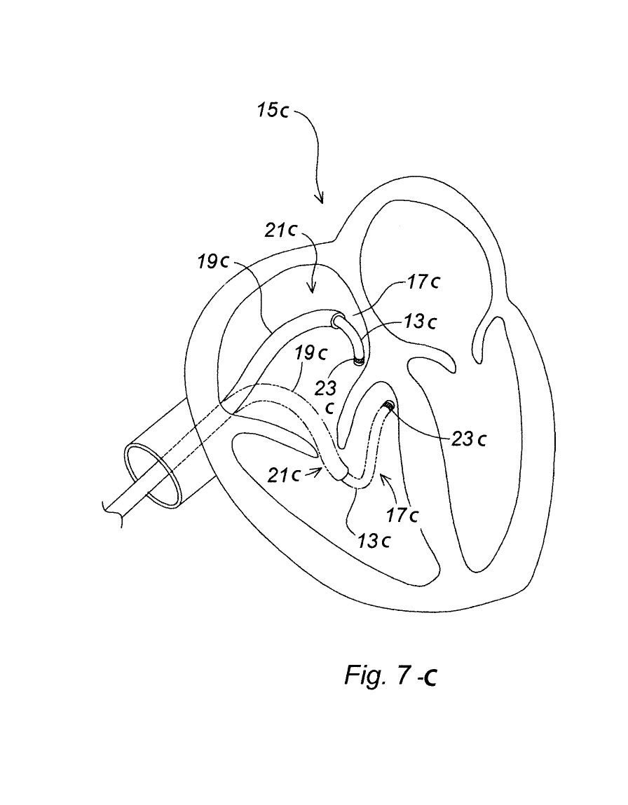

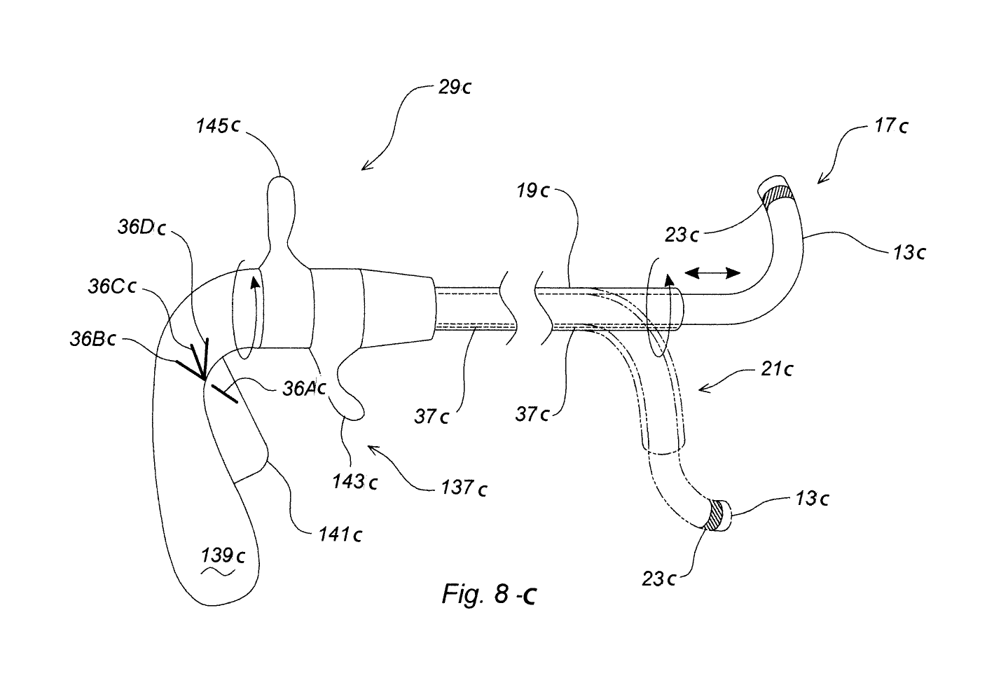

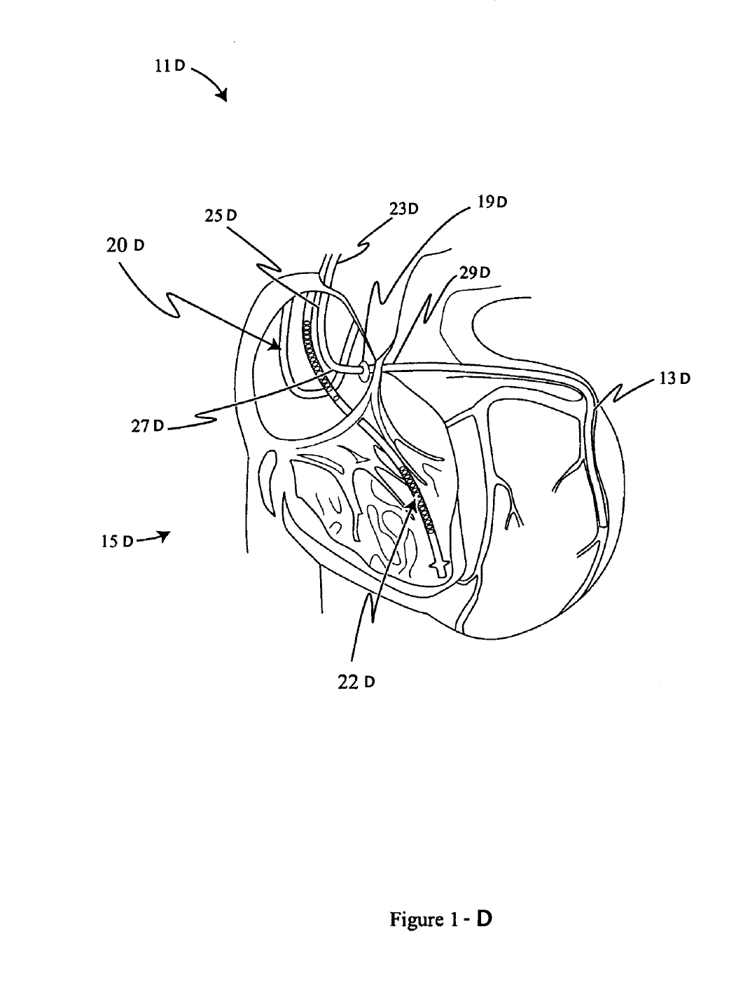

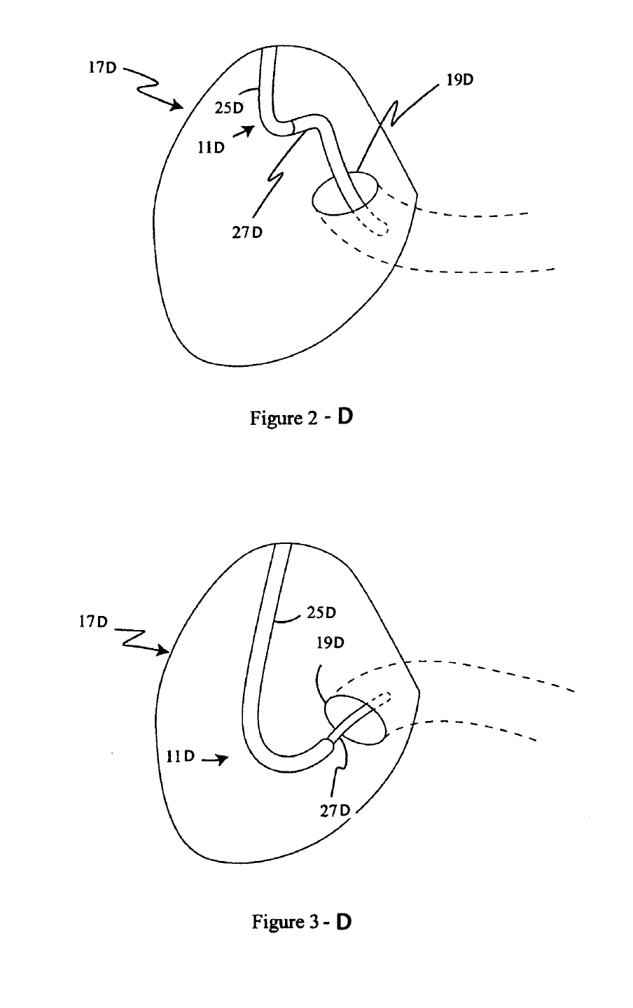

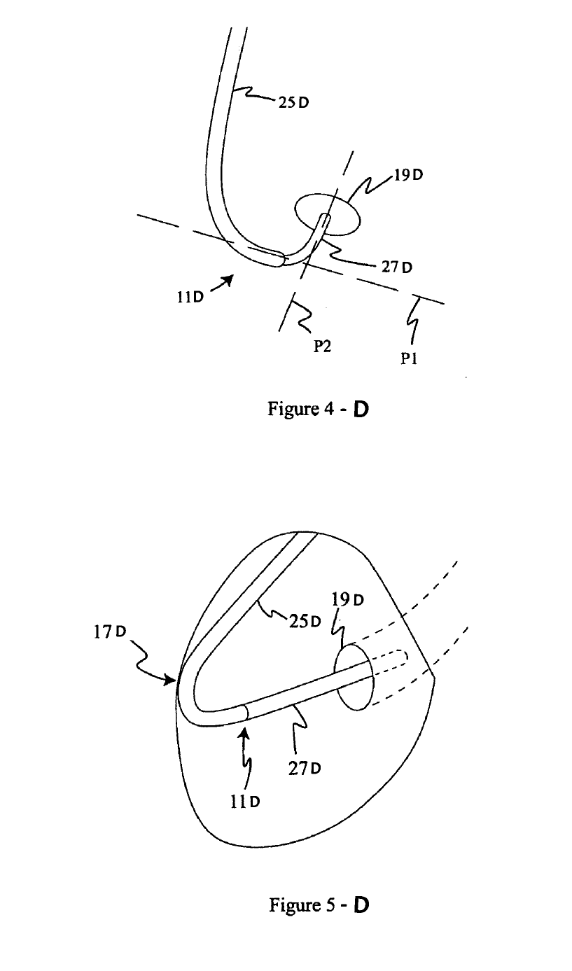

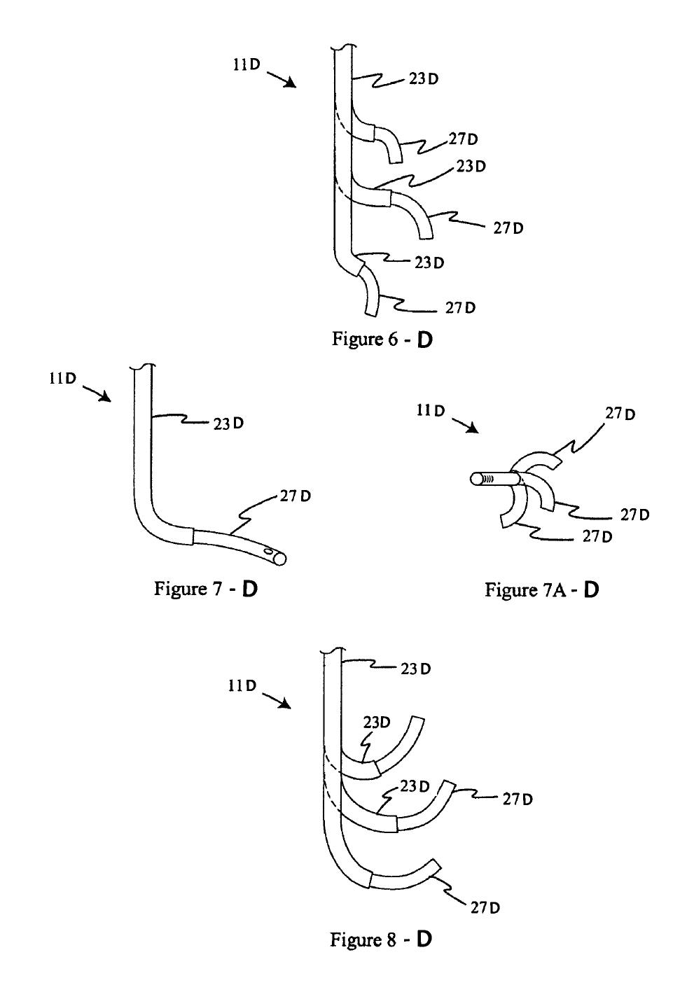

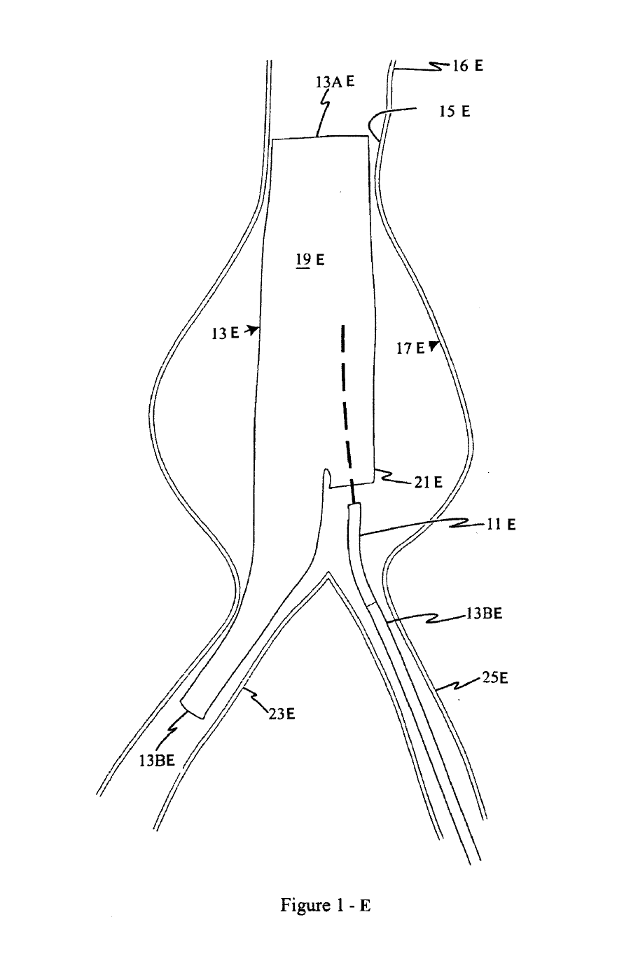

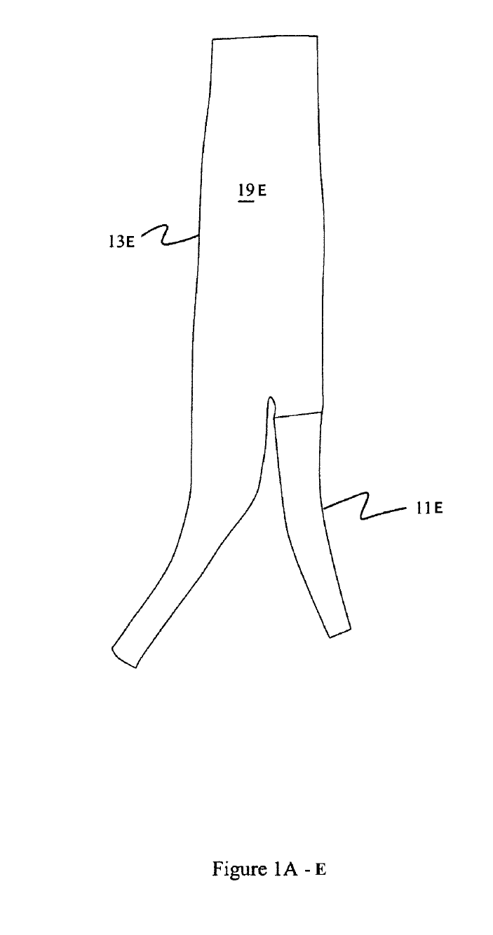

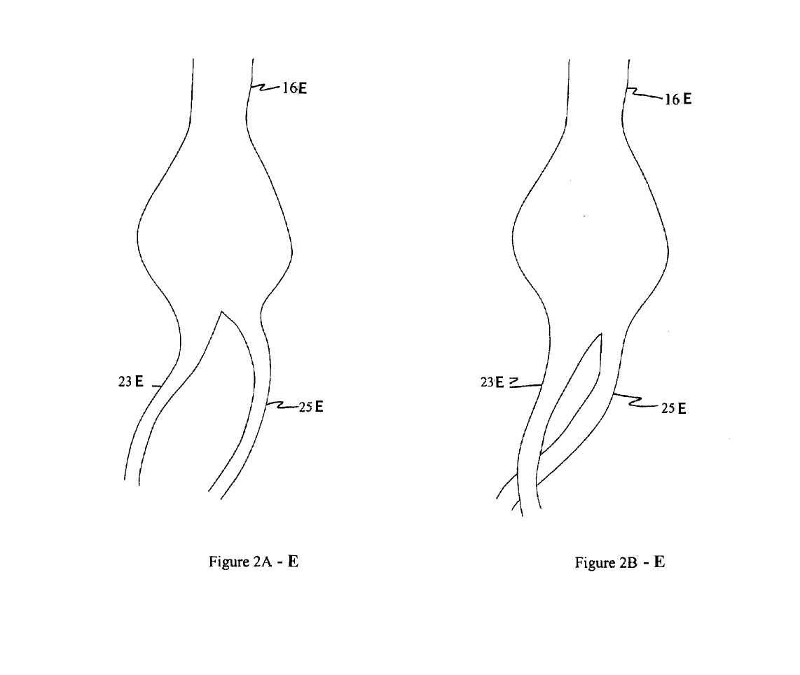

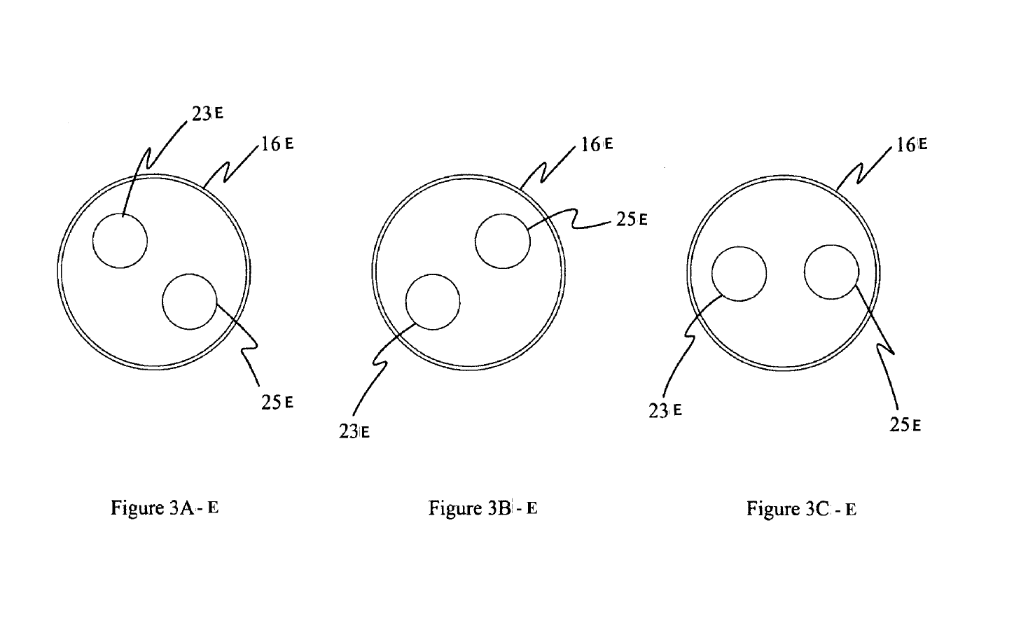

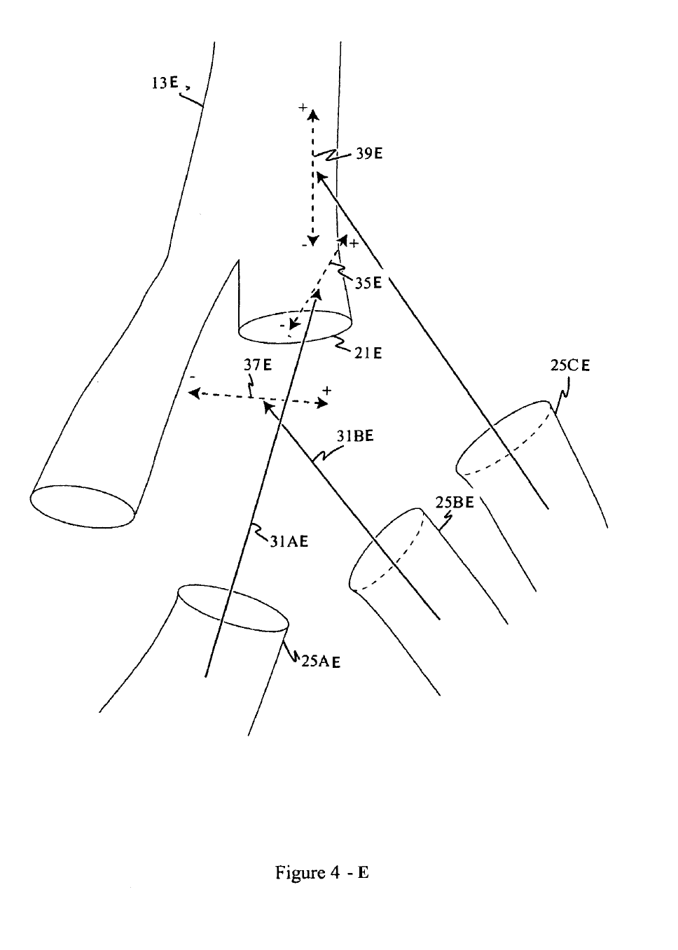

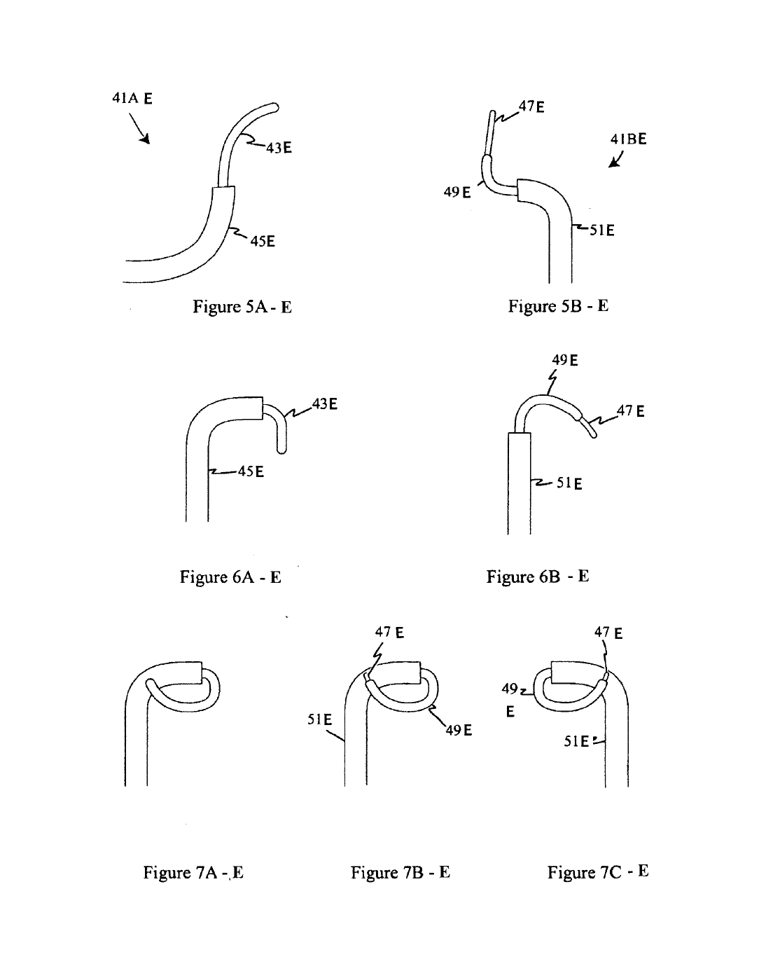

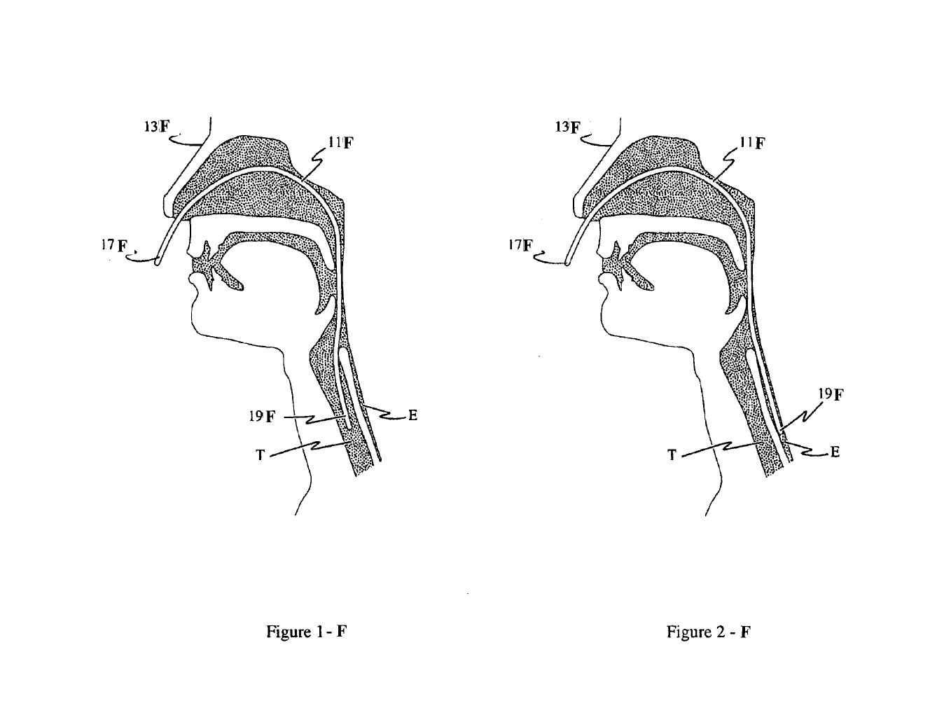

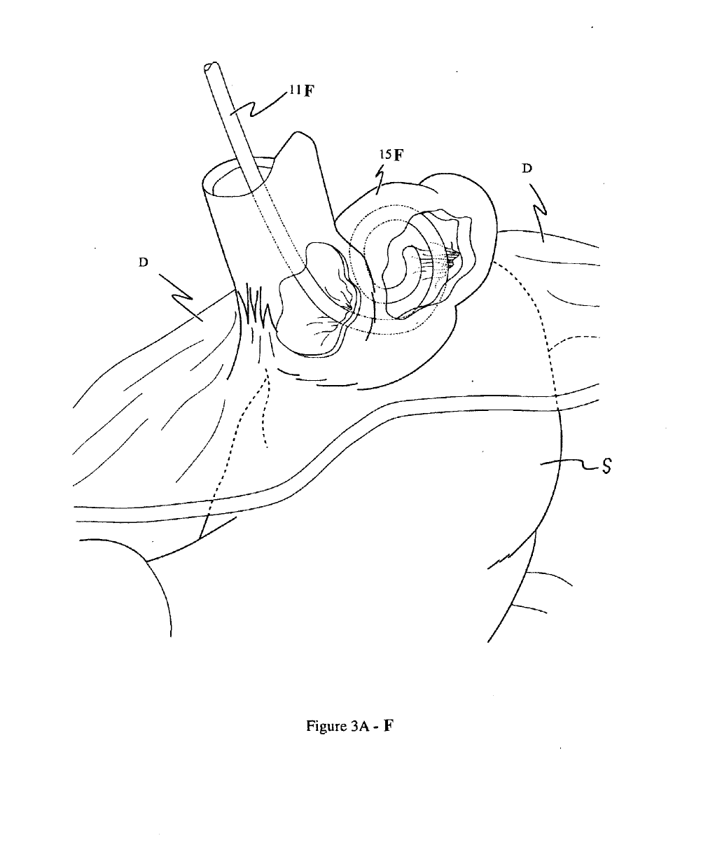

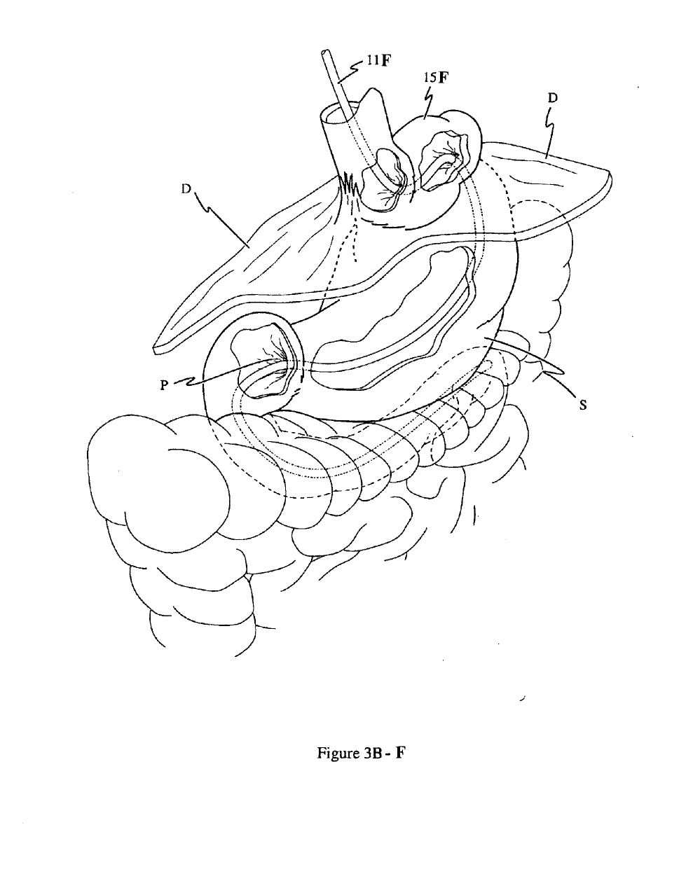

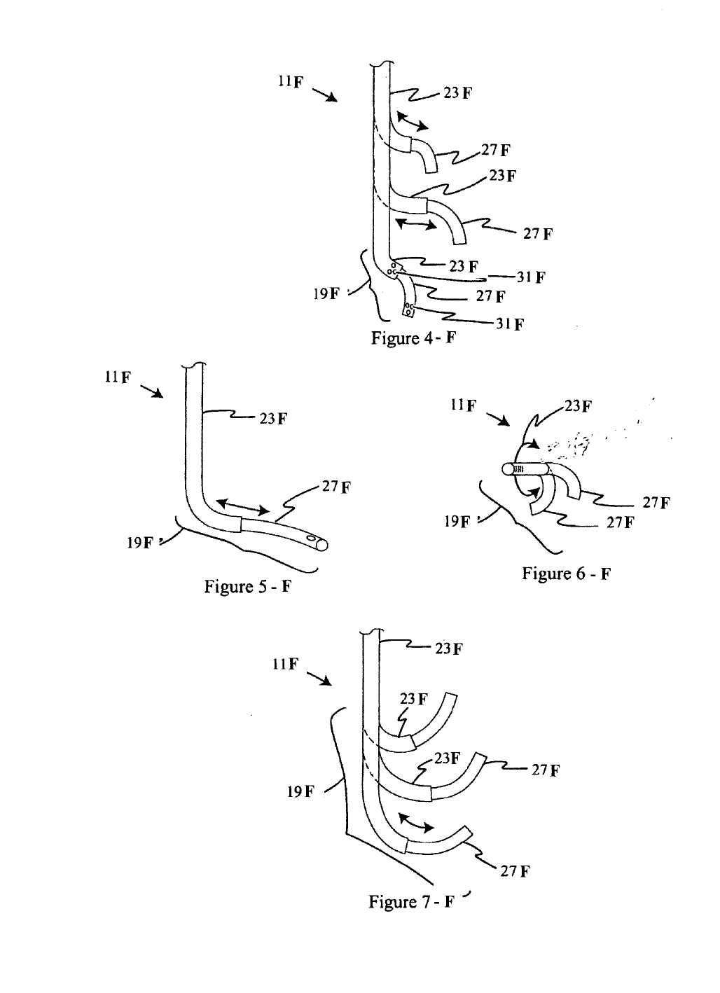

FIG. 4-C is a cross-sectional view of one embodiment of the catheter system illustrating one of a plurality of out-of-plane shapes formable with the present invention;

FIG. 5-C is a diagram illustrating that up-going and down-going shapes can be generated by one embodiment of the catheter system by a simple rotation of the medical element with respect to the catheter;

FIG. 6-C is a perspective view illustrating indicia which may be used to indicate to the user of one embodiment of the catheter system what type of curve is being generated in the distal end portion the system;

FIG. 7-C is a cross-sectional view illustrating forming shapes to map or ablate both above and below a heart valve annulus when using one embodiment of the present invention;

FIG. 8-C is a front elevation of an exemplary hand grip controller for one embodiment of the present invention; and

FIG. 1-D is a perspective drawing illustrating the use of the coronary sinus catheter system and method of the present invention in the human body.

FIG. 2-D is a front elevation showing an illustrative first shape into which the coronary sinus catheter system may be formed in connection with its use with the pacing lead.

FIG. 3-D is a front elevation showing an illustrative second shape into which the coronary sinus catheter system may be formed in connection with its use with the pacing lead.

FIG. 4-D is a front elevation showing an illustrative third shape into which the coronary sinus catheter system may be formed in connection with its use with the pacing lead.

FIG. 5-D is a front elevation showing an illustrative fourth shape into which the coronary sinus catheter system may be formed in connection with its use with the pacing lead.

FIG. 6-D illustrates various possible up-going configurations of the coronary sinus catheter of the present invention.

FIG. 7-D illustrates the basic out-of-plane shape achievable with the coronary sinus catheter of the present invention.

FIG. 7A-D illustrates various possible out-of-plane shapes of the coronary sinus catheter of the present invention.

FIG. 8-D illustrates various possible down-going configurations of the coronary sinus catheter of the present invention.

FIGS. 1-E and 1A-E are elevations showing the placement of a conventional stent graft.

FIGS. 2A-E and 2B-E illustrate some possible variation in the orientation of blood vessels in a living being.

FIGS. 3A-E through 3C-E are plane slices also illustrating such variation.

FIG. 4-E is a plane view illustrating misalignment in three dimensions of a vessel with the body of a stent graft.

FIGS. 5A-E and 5B-E are elevations showing up-going configurations of the guide element of the present invention.

FIGS. 6A-E and 6B-E are elevations showing down-going configurations of the guide element of the present invention.

FIGS. 7A-E through 7C-E are elevations showing out-of-plane configurations of the guide element of the present invention.

FIG. 1-F is a plan view of a nasogastric tube of the present invention, showing incorrect placement.

FIG. 2-F is a view similar to FIG. 1-F showing correct placement in the esophagus.

FIG. 3A-F illustrates improper placement due to an hiatus hernia.

FIG. 3B-F illustrates possible difficulties in placement into and/or through the stomach.

FIG. 4-F illustrates families of up-going shapes for the nasogastric tube.

FIGS. 5-F and 6-F illustrate families of out-of-plane shapes for the tube of the present invention.

FIG. 7-F illustrates families of down-going shapes for the tube of the present invention.

Corresponding reference numerals indicate corresponding parts throughout the several views of the drawings. The drawings described herein are for illustrative purposes only of selected embodiments and not all possible implementations, and are not intended to limit the scope of the present disclosure.

While one embodiment of the present invention is illustrated in the above referenced drawings and in the following description, it is understood that the embodiment shown is merely one example of a single preferred embodiment offered for the purpose of illustration only and that various changes in construction may be resorted to in the course of manufacture in order that the present invention may be utilized to the best advantage according to circumstances which may arise, without in any way departing from the spirit and intention of the present invention, which is to be limited only in accordance with the claims contained herein.

### DETAILED DESCRIPTION OF AT LEAST ONE PREFERRED EMBODIMENT OF THE INVENTION

In the following description, numerous specific details are set forth such as examples of specific components, devices, methods, in order to provide a thorough understanding of embodiments of the present disclosure. It will be apparent to a person of ordinary skill in the art that these specific details need not be employed, and should not be construed to limit the scope of the disclosure. In the development of any actual implementation, numerous implementation-specific decisions must be made to achieve the developer's specific goals, such as compliance with system-related and business-related constraints. Such a development effort might be complex and time consuming, but is nevertheless a routine undertaking of design, fabrication, and manufacture for those of ordinary skill.

In preferred embodiments of the present invention, the catheter system includes a catheter and sometimes a medical element also sized to pass through blood vessels to a heart of a patient. It is understood that the patient may be human or non-human. The medical element is generally configured to have an end portion that can be manipulated to assume and maintain various curved configurations that can assist the catheter system as it is routed through the patient, and that allows a medical device attached at the end portion of the medical element to be positioned and operated at the appropriate location within the heart. In alternative embodiments, the medical device may be disposed on one either the catheter or the medical element. Yet other embodiments of the present invention can be adapted for use in placement of pacing wires in the coronary veins of a heart, placement of feeding tubes or other nasogastric or nasoenteric tubes in a living being, and for colonoscopies.

Those of skill in the art understand that routing a catheter through the interior of the body of a patient is a complex task. The complexity of the routing procedure is greatly increased because of the three dimensional shape of the patient's body, internal organs, and vessels. As such, this means that at any point in the catheter routing process, the catheter may need to be routed straight ahead, to the left, to the right, or even reversed upon itself in order to route the catheter through vessels and obstructions found in the patient's body. In fact, if the tip of the catheter is thought of as the inside center point of a sphere, the tip of the catheter may need to be routed in the direction of almost any point on the outer surface of the sphere in order to get the catheter through the vessel or obstruction and to place the tip of the catheter where it is needed. Thus, as the catheter system is routed and used within the patient, certain preferred embodiments of the present invention include the ability to curve the catheter and the medical element into various configurations.

In defining this wide range of curvatures, the disposition of the medical element can be defined in terms of the geometric relationship as it relates to disposition of the catheter. More specifically, it will be understood that in the present embodiments, the catheter system includes a catheter that cooperates operationally with a medical element. The catheter is preferably a substantially straight and cylindrically-shaped tube that is flexible enough to be curved into an arc. When the catheter is curved into a single arc, the axial centerline of the catheter tube is curved to define a first plane. A geometric plane, of course, requires three points to be properly defined, and here, the three points consist of: (1) the point where the axial centerline of the substantially straight catheter tube begins to curve; (2) the point where the curvature of the axial centerline of the substantially straight catheter tube ends: and, (3) any point on the curved axial centerline of the substantially straight catheter tube when the catheter tube is curved.

In a similar manner, the medical element is also generally a substantially straight, cylindrically-shaped, flexible tube that, when curved, defines a second plane. Thus, the separate curves of the catheter tube and the medical element tube result in separately defined planes. It will be appreciated by those skilled in the art that the cooperative association between the first plane of the catheter and the second plane of the medical element is intended to achieve a level of manipulation necessary to allow some preferred embodiments of the catheter system to be routed and positioned within the heart of an animal for execution of the desired medical procedures. In alternative embodiments of the present invention, the catheter system may be adapted for use as an endoscope or a colonoscope. In fact, it will be understood that the various embodiments of the present invention can be readily adapted for any use, and still remain within the intended scope of the claims, where such use requires the insertion of a catheter into the body of an animal and the catheter needs the ability for the catheter system or its components to be shaped into the various curves and configurations as described herein.

Within the various embodiments of the present invention, the most common geometric configurations for the catheter system are: (1) a down-going configuration; (2) an up-going configuration; and, (3) and an out-of-plane configuration. A down-going configuration occurs as shown in FIG. 3A-C through 3C-C where the catheter system has been placed into a shape in which the tip of the catheter system points in a direction opposed to the direction in which the catheter system would move if it were moved as a unit farther into the patient. An up-going configuration is, of course, the opposite of the down-going configuration and occurs when the catheter system has been placed into a shape in which the tip of the catheter system points in a direction that is the same as the direction in which the catheter system would move if it were moved as a unit farther into the patient.

It will be understood that the up-going and down-going configurations usually exist in a two-dimensional plane. The patient's body, however, is a three dimensional object and the catheter system's configurations need to also be three dimensional. Therefore, the out-of-plane configuration occurs when the first plane, as formed by the curved catheter tube, is not coplanar with the second plane, as formed by the curved medical element tube. An example of an out-of-plane configuration can be shown in FIG. 4-A.

It is important no note that preferred embodiments of the present invention can generate all of the above-noted up-going, down-going, and out-of-plane configurations in an empty three dimensional area by only the manipulation of the catheter system controls disposed outside the patient's body. This is to say, the configurations can be generated without the need for any component or element of preferred embodiments of the present invention to be braced against any other element or component, or any portion of a patient's tissue, organ, bone, or element of the patients body. This unique ability distinguishes this present embodiment from previous catheter inventions that are unable to be configured into the above-noted configurations without bracing at least one element of those previous catheter inventions against another element or component, or any portion of a patient's tissue, organ, bone, or element of the patient's body. This allows preferred embodiments of the present invention to overcome the many medical issues that can arise when the inner tissues, vessel, bones, and organs of a patient's body become unexpectedly tortuous or abnormally configured without having to spend an excessive amount of time in attempting to manipulate the catheter through those tortuous areas and without having to remove the catheter to install various accessories to the tip of the catheter to allow the catheter to be routed through the tortuous portions of the patient's body.

A basic embodiment of the present catheter system invention is disclosed in FIGS. 1A-A through 1C-A. A catheter 31A (see FIGS. 1A-A through 1C-A) of the present invention in its simplest form includes an enabling sheath 33A, and an inner catheter 35A having a distal tip with a complex-curve shape. Catheter 31A is especially suited for selective arterial catheterization. In that application, the catheter is custom shaped or formed while in the patient to make it easy to direct the tip in any orientation required to enter a branching orifice or serpiginous vessel. Catheter 31A is not a steerable catheter, but rather one which may be custom curved and recurved by the user to select each branch vessel. The catheter may also be used in the biliary tree and urinary tract to negotiate branches, corners, and serpiginous pathways.

As will become apparent in view of the following disclosure, manipulation of catheter 31A results in mimicking virtually any simple or complex curved configuration of selective arterial catheter shape imaginable while the catheter is disposed in the patient Likewise, modification of curve and tip orientations allow selection and direction of wire guides for other invasive procedures such as percutaneous cholangiography and percutaneous nephrostomy; that is, any procedure requiring direction or redirection of a catheter.

Inner catheter 35A is a complex memory curve catheter which runs in a coaxial manner through enabling sheath 33A. With the complex tip completely extended beyond the sheath, the most complex tip configuration reforms. By pulling the inner catheter back through the enabling sheath to varying degrees (sheathing and unsheathing the inner catheter tip), various segments of the curve are “ironed out”—thereby changing the overall catheter tip configuration and tip orientation. The inner catheter is the active primary component but is a passive passenger with respect to the enabling sheath which acts upon the catheter to modify its shape. In addition, the fact that the transformation takes place in a vessel in the human body further modifies the shapes which can be achieved due to interaction of the catheter system with the walls of the vessel sheath 33A has the capability of being formed by a pullwire 37A into a hook configuration, as described below. (Although described as a wire, item 37A could equivalently be made of high tensile strength suture or thread material.) This capability allows reformation of the complex memory curvature by directing the catheter tip downward while catheter system 31A is disposed in the aorta. Secondarily the sheath curve can, to some degree, act on the catheter to further modify the catheter shape.

In detail, catheter system 31A (see FIGS. 1A-A through 1C-A) includes outer enabling sheath 33A which extends almost the entire length of the catheter. It is preferred that sheath 31A be long enough so that only the complex curve distal end portion of the inner catheter extend beyond it. Inner catheter 35A is disposed in the central bore of sheath 33A and has the most exaggerated sidewinder preformed tip configuration distally. (Of course, any other similar complex-shaped memory curve inner catheter could also be used in the present invention as inner catheter 35A.)

The distal end of enabling sheath 33A can be formed into a curve with up to one hundred and eighty degrees of curvature when retracted by pull wire 37A by the suitable application of tension to the pull wire. This allows the hook configuration of the sheath 33A to reform the catheter 35A when the catheter is advanced through the sheath. The variable curved tip also allows variation in the degree of curvature, modifying the natural memory curve and thereby the overall shape of the catheter.

Enabling sheath 33A modifies the extreme natural curvature of catheter 35A by acting as a housing that irons out various segments of the curvature when the catheter is retracted back into the sheath. Such an “ironing” effect is illustrated in FIG. 2A-A. In FIGS. 2-A and 3-A, the various portions of catheter system 31A are shown in simplified form for clarity. For example, in FIG. 2A-A, the catheter 35-A is shown extending distally from sheath 33A a small amount, while catheter and sheath are disposed in a vessel (such as an artery) 41A.

In FIG. 2B-A, the same catheter 35A is shown extended distally out of sheath 33A a small additional amount such that the catheter regains some of its curvature. Similarly, in FIGS. 2C-A and 2D-A, catheter 35A is extended distally even further. Note that in FIG. 2D-A, the distal curved portion of catheter 35A is fully extended from sheath 33A, but the original configuration of catheter 35A is not obtained because of the interaction of the catheter with vessel wall 41A.

If the physician desires to instead shape or form catheter 35A back into its original, preformed shape of FIG. 1-A, the steps illustrated in FIGS. 3A-A through 3C-A are followed. In this case, the sheath is first formed (by use of retraction wire 37A) into the shape shown in FIG. 3A-A and FIG. 3B-A. As a result of this shape of the sheath, as catheter 35A is moved distally with respect to the sheath it assumes the form in the vessel as illustrated in FIGS. 3A-A and 3B-A. The sheath is restraightened by suitable manipulation (i.e., release) of retraction wire 37A as shown in FIG. 3C-A as desired. This procedure allows the original form of catheter 35A to be obtained in the vessel.

Note, from comparing FIG. 2D-A with FIG. 3C-A, the great differences in catheter shape achievable by simple manipulation of sheath 33A and of the catheter with respect to the sheath. In fact FIGS. 2A-A through 2D-A and 3A-A through 3C-A all illustrate some of the multitude of different catheter shapes which may be formed in the body, during a medical procedure, using the present invention. It should be realized that these shapes are merely illustrative and that with suitable manipulation of the sheath and relative movement between the sheath and the catheter, a great number of additional catheter configurations may be achieved.

Note as well, that the particular configuration shown in FIG. 3B provides a tighter radius for the tip of catheter 31A than is achievable with prior devices due to the interaction of complex-formed tip of the inner catheter and the deflection of the sheath. This greater flexibility in the shapes achievable permits vessels to be entered with the present catheter which were not readily accessible with the prior devices.

Referring back to FIGS. 1A-A through 1C-A, the inner catheter 35A travels through the enabling sheath in a coaxial manner, but is stiffened from its proximal end to a point just proximal to the distal exaggerated curvatures by an outer stiffening segment 43A. This outer stiffening segment is fused to the inner catheter proximally and distally and provides sufficient strength to the inner catheter to permit it to be moved axially with respect to the sheath without collapsing or binding.

Catheter system 31A is introduced in the usual manner through the femoral artery and advanced into the abdominal aorta. The catheter is reformed and selectively shaped in the abdominal aorta. When initially introduced the inner catheter is in its ironed parked position within the enabling sheath with only a small distal tip segment protruding (see FIG. 2A-A). The pull wire 37-A is retracted, forming a hooked curve (see FIG. 3A-A) which allows direction of the catheter tip downward and thereby easy reformation of the memory curve. Once reformed, the various shapes of the catheter can be selected by advancing or retracting the catheter into the enabling sheath, thereby allowing or disallowing the natural memory curves to form at various segments. Pull wire modification of the enabling sheath allows additional curved configurations.

Proximally, the stiffened inner catheter is pulled back through a plastic sleeve 45, slotted on either side. (See FIGS. 1C-A and 1D-A.) A fixation collar 47A attaches to the proximal portion of the catheter. A molded spring “V” prong 49A, rounded on one side and flat on the other, arises from the side of the collar and extends through the sleeve. The prong is fixedly secured to catheter 35A and can be pulled through a right slot 51A of the sleeve (see FIG. 1D-A), thereby pulling the catheter as a whole through the enabling sheath and ironing out various degrees of memory curve in the distal tip of the catheter. The spring “V” prong 49A when squeezed together passes freely through the slot whose border is straight inferiorly but serrated superiorly as shown in FIG. 1D-A. When released the prong springs open and the rounded segment locks in a chosen serrated position. Distally, a crossing slot 53A allows the prong to be pulled across to the left slot (similar in size and shape to the right slot 51A shown), thereby twisting and torquing the entire catheter one hundred and eighty degrees so that when the prong 49A is pulled back in the left slot position the curved configuration is ironed out, resulting in additional variations in catheter shape. The locking pattern of the left slot is opposite to that of the right—flat upper border, serrated lower border, still allowing collar locking in various retracting positions.

The inner catheter proximally must exit through a valve (such as hemostasis valve 61A shown in FIG. 1C-A) as found in current arterial sheaths. A “Y” branch side port 63A allows constant pressure flushing of the sheath to prevent possible clot formation and allows water activation of the hydrophilic retraction wire.

On top of the side port 63A, slide tracks 65A (see FIG. 1E-A) are molded. The tracks contain a plastic “S” spring slide 67A with locking teeth. The slide spring is attached to the pull wire 37A proximally. As the slide spring is disengaged by pushing downward and pulling back, the wire is retracted. When released the “S” slide locks into the selected position in the track. When straightening of the enabling sheath is desired, the slide 67A is disengaged and pushed to the forward position, thereby releasing traction on the wire and allowing the enabling sheath to reform spontaneously or with help by sliding the reinforced catheter towards the tip or by placing a straightening wire guide through the inner catheter lumen.

The ventral primary curvature segment of the enabling sheath is biased (as indicated at the reference numeral 71A, FIG. 1A-A) to curve in a desired direction by making the sidewall along the inner portion of the desired curve more flexible due to the type or thickness of the material in this segment. Similarly, a flexibility bias can be established by designing exposed or covered gaps along one side of a segment of the sheath over which the bend is desired. When pulled distally, the more flexible or gapped side of the segment will give first, thereby, allowing curvature in a precisely selected segment and direction.

In view of the above, it will be seen that the various objects and features of the present invention are achieved and other advantageous results obtained. The examples of the present invention disclosed herein are intended to be illustrative, and

In an alternative preferred embodiment of the present invention, the formability of the catheter system components can be achieved by the incorporation of certain “weaknesses” in the catheter system elements. FIG. 4AB shows a sheath or catheter 72B of the present invention includes a medical tube 73B having an exterior wall and an internal lumen extending substantially through the length of the tube. The tube has an outside diameter sufficiently small so that it may be inserted into the human body. The distal end 74B of tube 73B is shown in FIG. 4A-B.

Wire 37B runs from the proximal end of the tube to the vicinity of the distal end of the tube, and, in the same manner as described above in connection with FIG. 1-B, is connected to the tube adjacent the distal end so as to allow the distal tip of the tube to be deflected upon movement of the wire. The tube has a substantially uniform transverse cross section throughout a substantial part of its length, except for the distal tip section. The tube wall adjacent the distal end has gaps 75B, 77B, and 79B therein on one side of the longitudinal axis of the catheter tube so as to form a predetermined region of weakness 71B. As a result, upon tension being applied to the wire the distal end of the catheter tube bends in the direction of the predetermined region of weakness.

Although gaps 75B, 77B, and 79B can all be the same size, it is preferred that they differ in size as shown in FIG. 4A-B. The three gaps of FIG. 4A-B differ in depth, as can readily be seen. It has been found that with this construction the catheter or sheath preferentially bends first at the gap with the greatest depth (gap 79B), and next at the gap with the second greatest depth (gap 77B), and finally at the gap with the least depth (gap 75B), as shown in FIGS. 4B-B through 4D-B, as increasing tension is applied to wire 37B.

Although gaps 75B, 77B, and 79B of FIG. 4-B are generally crescent moon shaped, the present invention is not limited to any particular gap shape. For example, gaps 81B having a generally diamond shape (FIG. 5-B), or gaps 83B having a compound arrowhead shape (FIG. 6-B), or any of a number of other gap shapes may also be used. Gaps shaped so that portions of the catheter tube wall on opposite sides of each gap may overlap when the catheter is formed by the wire to have its greatest curvature may be used, as may gaps shaped so that there is no overlap (although overlapping can provide a tighter curve on the distal end of the sheath/catheter). Similarly, gaps shaped so that the distal wall portion overlaps the proximal wall portion of the gap when the catheter is formed by the wire to have its greatest curvature may be used, and may be gap shaped so that the proximal wall portion overlaps the distal wall portion of the gap.

Nor is there any rigid requirement concerning depth of gap. Although the gaps of FIG. 4-B extend through the walls of the sheath/catheter to the lumen, that is not a requirement. In FIG. 7-B, for example, a gap 75AB is provided which terminates short of the sheath/catheter lumen. It still helps provide the desired predetermined region of weakness 71B. Also shown in FIG. 7-B is a reinforcing section 85B which may, for example, be co-extruded with the sheath/catheter. This reinforcing section may be used in combination with the gaps, or by itself, to cause the distal end of the sheath/catheter to bend in the direction of region 71 B when tension is applied to wire 37B.

No matter what the shape of the gap, it is preferred that the inner surface of the gap adjacent the lumen of the catheter be smooth.

For some applications, it may be preferred that the gaps be covered. FIG. 8-B shows a layer of material 87B covering the gaps. It should be realized that the physical appearance of layer 87B in FIG. 8-B is illustrative only. The appearance will differ depending upon how layer 87B is made. It can be formed by dipping, fusing, or heat shrinking, for example.

Similarly, the gaps themselves may be formed in a number of different ways. They can be laser cut, water jet cut, punched, or formed by any other suitable procedure.

In addition, various ways can be used to form the predetermined region of weakness 71B, either alone or in combination with the gaps. For example, FIG. 9-B shows a region of weakness 71B formed by reducing the thickness of the sheath/catheter wall at the predetermined region of weakness. Or, the predetermined region of weakness may be formed by having a portion of the wall which is chemically different from the tube wall elsewhere, or otherwise differs in some way from the rest of the sheath/catheter wall. This is accomplished, for example, by leaching material out of the appropriate portion of the sheath/catheter wall to form the region of weakness, or by using a different material in that region which is weaker than the material which makes up the rest of the sheath/catheter wall.

It should be appreciated that when gaps 75B, 77B and 79B are cut into a straight sheath/catheter, and the layer 87B put over those gaps, there is the possibility that layer 87B material will bulge into the inner lumen of the sheath/catheter when the distal end is curved by application of tension to wire 37B.

This bulging can be prevented if the distal end of the catheter tube is preformed into a curved shape with the gaps on the inner side of the preformed curve, as shown in FIG. 10-B. The gaps are then in their “rest position” when the distal end of the sheath/catheter is fully curved. A stretchable covering material 87B is then deposited over the gaps in this curved, “rest” position, so that the stretchable covering layer is substantially relaxed when the tube is disposed in a predetermined bent position.

With this construction, the stretchable covering layer 87B is substantially in tension when the distal end of the sheath/catheter is straight, and the covering layer tends to pull the distal end back to the preformed, curved shape. To overcome this tendency, a spring (or other suitable device) 91B is provided for biassing the stretchable covering layer into a stretched position, as shown in FIG. 11-B. A jacket of material 93B is provided to cover the biassing spring.

Turning to FIGS. 12-B and 12A-B, it is desirable to make the catheter system of the present invention as easy to use as possible. To facilitate this, a handle assembly 95B is provided which is an alternative to the structure shown in FIGS. 1C-A through 1E-A. Handle assembly 95B includes a handle 97B fixedly secured to sheath 33B. Handle 97B has a slot 99B formed therein in which rides a thumbwheel operated clamp 101B (shown in more detail in FIG. 12A-C). The body of the thumbwheel operated clamp is connected to wire 37B, so that the user may manually adjust the tension on wire 37B by moving clamp 101B to the desired position in slot 99B and fix the clamp in that position to maintain the desired tension on the wire. Since the position of clamp 101B in slot 99B is infinitely variable, the tension on wire 37B is also continuously variable over the desired range.

The construction of FIG. 12-B also provides for infinite variation in the rotational and longitudinal positioning of catheter 35B with respect to handle 97B. The handle has secured thereto a spring clamp 103B. Catheter 35B may be held by the jaws of spring clamp 103B (or other suitable device) in any desired rotational or longitudinal position, as controlled by the user. The jaws of the clamp are merely opened to allow the user to move the catheter longitudinally and/or rotationally, and then closed on the catheter to hold it in the new desired position.

Also shown in FIG. 12-B is an auxiliary line 105B which constitutes means (in combination with a syringe, for example) for forcing fluid between the sheath 33B and the catheter 35B. Such flushing helps to prevent the formation of clots, for example.

As is noted above, it will understood by those of skill in the art that the above embodiments of the catheter system can be readily adapted to generate alternative embodiments to fit certain medical procedure and applications as suggested herein. Some of those preferred embodiments are described in the following paragraphs.

For example, in one alternative embodiment of the present invention, the basic catheter system can be equipped with a medical tool such as, for example, an ablating tool for ablating tissue from an organ or a heart mapping tool for mapping the internal region of a heart, is disposed at the distal end of at least one of either the catheter or the medical element.

Turning to the drawings at FIG. 1-C through 8-C, a preferred exemplary embodiment of a catheter system 11C is shown including a medical element 13 sized to pass through blood vessels to a heart 15C of a patient. The medical element has a distal end portion 17C capable of assuming and maintaining a curved configuration as shown in FIG. 1-C through 8-C. A catheter 19C is disposed with respect to the medical element so as to permit manipulation of the medical element 13C. The catheter 19C is capable of assuming and maintaining curved configurations such that the distal end portion of the catheter system 11C may be sequentially formed into up-going, down-going, or out-of-plane configurations as needed. In certain embodiments, it is preferred that the distal end portion 21C of the catheter be capable of assuming and maintaining the curved configurations so that the curvable distal end portions 17C and 21C may interact. Depending upon the type of medical procedure to be performed, a medical tool is disposed at the distal end of at least one of either the catheter or the medical element. In certain embodiments of the catheter system, the medical element is disposed within the inside of the catheter in an annular arrangement. In other embodiments, the catheter and the medical element may be in a side-by-side arrangement such that the outer surfaces of the catheter and the medical element are in fixed or non-fixed contact.

In one preferred embodiment, the medical tool is an ablation tool 23C (FIG. 2-C) that is disposed at the distal end portion of at least one of the medical element 13C and the catheter 19C for ablating tissue in the heart when the distal end portion of the catheter system is disposed in the heart 15C. Ablation tools may include cryo-surgery tools (see tool CT in FIG. 2A-C-note that loop cryo-surgery tools may also be used), microwave tools (microwave antenna MA in FIG. 2B-C), radiofrequency tools (electrode 23C in FIG. 2-C), and/or laser tools (tool LA in FIG. 2C-C), all of which are known in the art. See U.S. Pat. No. 5,733,280 for cryo-surgery ablation tools, U.S. Pat. No. 5,370,678 for microwave ablation tools, and U.S. Pat. No. 6,283,955 for laser ablation tools, the disclosures of which are incorporated herein by reference. Such tools are well-known and these are given for illustrative purposes only.

In the present embodiment, an ablation source may be connected to the ablating tool. The ablation source is normally dependent upon the type of ablation tool used to perform the ablation procedure. For example, an ablation source for cryo-surgery ablation tools is a source of low temperature fluid for passing to the ablation tool, an ablation source for laser ablation tools is a source of laser energy for transmission to the laser ablation tool, an ablation source for a microwave ablation tool is a source of microwave energy for application to the microwave ablation tool, and an ablation source for a radiofrequency ablation tool is a source of radiofrequency power for supplying to the radiofrequency ablation tool.

When an ablation tool is used with the catheter system, the method of ablation includes manipulating an electrophysiology catheter embodiment of the present invention and includes curving an electrophysiology catheter into a desired shape, bending the distal end portion of a catheter disposed in a predetermined relationship with respect to the electrophysiology catheter to fix the electrophysiology catheter in a desired position in a heart, changing the relative positions of the electrophysiology catheter and the catheter either translationally or rotationally to change the position of the electrophysiology catheter in the heart, and ablating or mapping a portion of the heart based upon the position of said electrophysiology catheter in the heart.

In alternative embodiments where mapping of the heart is needed, the ablation tool is replaced with a heart mapping tool. A common mapping tool usually includes an electrode of some type that can locate and identify electrical impulses within the heart. In this embodiment, at least one mapping electrode is disposed at the end portion of at least one of either the catheter or the medical element shaft for mapping electrical signals in the heart when the distal end portion of the catheter system is disposed in said heart. A receiver for accepting communications from the mapping electrode can also be included.

In yet other alternative embodiments, the catheter system uses neither an ablation tool or a mapping tool, but is instead any medical tool that must be routed internally through the body of the patient and the manipulation of the catheter system forms a substantially stable platform in a cavity of the heart from which a medical tool may be extended and used. It is understood that the medical tool used with the catheter system may either be pre-attached to the distal end of at least one of either the catheter or the medical element, or the medical tool can routed through components of the catheter system for placement of the medical tool at the position needed for the selected medical procedure, with the final placement of the medical tool occurring after the catheter system has been placed and positioned in accordance with the descriptions herein.

Medical element 13C (FIG. 3A-C through 3C-C) is movable translationally (longitudinally) with respect to catheter 19C. When the distal end portions of one or both of the medical element 13C and catheter 19C are curved, relative longitudinal movement results in varying the shape of the distal end portion of the catheter system 11C, as shown in FIG. 3A-C through 3C-C. This feature allows the distal end portion of the present embodiment of the catheter system (made up of the distal end portions of both the medical element and the catheter) to form particular shapes as necessary to map or ablate particular tissues in the heart. In FIG. 3A-C through 3C-C, the shapes shown are all down-going configurations as defined above. Similarly, when medical element 13C (FIG. 4-C) is moved rotationally with respect to the catheter 19C, the medical element forms an out-of-plane configuration, also as defined above. Changing the rotational relationship between the catheter 19C and the medical element 13C can be used to change the shape from down-going to up-going, and vice versa, as shown in FIG. 5-C.

As shown in FIG. 6-C, it can be useful to include indicia 31C, 33C, 35C, etc. near the proximal end of the catheter system and near the control mechanism 29C to assist the rotating and translating the medical element with respect to the catheter. Control mechanism 29C can include a first part 29AC which is fixed with respect to the catheter 19C and includes a reference indicium 31C. The control mechanism 29C can also include a second part 29BC which is fixed with respect to the medical element and includes several reference indicia 33C, 35C, etc. When indicia 31C and 33C are lined up the catheter 19C and medical element 13C curved portions are planar (this is also true when indicium 31C is lined up with indicia (not shown) on part 29BC which indicates a 180 degree orientation). The relative position of the indicia indicating anything other than 0 degrees or 180 degrees indicates to the user an out-of-plane configuration of greater or lesser degree. Similar indicia 36AC through 36DC (see FIG. 8-C), based upon the amount of curvature of the catheter and/or the medical element, can indicate whether the distal end portion of the system is up-going or down-going. It will be appreciated by those skilled in the art that while the indicia shown in the present embodiment are line segments, other types of indicia such as words, numbers, and symbols may also be used while remaining within the intended scope of the present invention. Likewise, it will be understood the certain embodiments of the present invention may also include methods of setting the control mechanism to at least temporarily fix the relationships between the catheter 19C and the medical element 13C as need to brace or maintain the medical tool in a substantially fixed platform or location to position or brace the medical tool when it is performing a medical procedure. When that specific medical procedure is completed, the fixation of the relationship between the catheter 19C and the medical element 13C can be released, and the catheter system can then be repositioned, readjusted, and re-fixated as needed to set up and perform the next medical procedure.

It should be appreciated that these various shapes can be selected and implemented to reduce trauma to the heart during mapping and ablation. Both the medical element 13C and the catheter 19C may be curvable by control elements actuable at the proximal end of the catheter system, or one or both of the medical element and the catheter may have a permanent curvature. Such control elements (like element 29C) for controlling pull-wire catheters are known. The system of the present invention is extremely versatile and is capable of mimicking the shape of any commercially available electrophysiology catheter. For example, (see FIG. 7-C) the catheter system 11C is shapeable into shapes to access the walls of the heart both above and below the tri-cuspid valve. Similarly, the catheter system is shapeable into shapes to access the walls of the heart both above and below the mitral valve, the adjacent the coronary sinus, or any other desired shape.

It should further be appreciated from the above, that shapes can be formed sequentially as needed to map and/or ablate various areas of the heart. Simple translation and/or rotation of the medical element 13C with respect to the catheter 19C result in a vast plurality of desirable shapes.

In an alternative embodiment, the catheter system can include a hand grip device adapted to be held by a human user. The hand grip device cooperates with other components of the catheter system to one of either: (1) adjust the shape of either the catheter 19C or the medical element 13C to result in either an up-going or down-going direction; (2) rotate one of either the catheter or the medical element; or, (3) rotate one of either the catheter or the medical element to modify the planar relationship between the first plane generated by the curve of the catheter and the second plane generated by the curve of the medical element wherein the planar relationship is either coplanar or out of plane. A lever or similar mechanism is provided for rotating the other of the medical element 19C and the catheter tube with respect to the hand grip such that the catheter tube is controllably rotated with respect to the medical element 13C. A knob, lever, or the like is also provided for curving the distal end portion of the catheter tube using a pull-wire. (See description below.) Using the hand grip, the catheter system 11C may be controlled to form up-going, down-going and out-of-plane shapes suitable for ablating and/or mapping tissue in a heart.

Turning to FIG. 8-C an exemplary hand grip control 137C is shown which is particularly suitable for one-handed control of the catheter system 11C. The hand grip control 137C includes a pistol-grip 139C adapted to be held in the hand of a user. A first trigger 141C mounted to the pistol grip 139C is suitably connected to a pullwire 37C or other suitable deflecting device to cause catheter 19C to curve (as desired by the user) to a curved position such as that shown in phantom in FIG. 8-C. A second trigger 143C mounted to the pistol grip is suitably connected to the medical element 13C to move the medical element translationally (as indicated by the double-headed arrow) with respect to catheter 19C when actuated by the user. Hand grip control 137C also includes a thumb actuable lever 145C to allow the user to rotate catheter 19C with respect to the handle and the medical element 13C. When lever 145C is moved by the user in the direction indicated by the arrow in FIG. 8-C, the catheter 19C is rotated with respect to the medical element 13C. Similarly, medical element 13C may be controlled by similar control mechanisms to advance and retract medical element 13C as well as curve the distal end thereof. As noted above, once the components of the catheter system have been routed and properly located, the hand grip control 137C can include elements that can temporarily fixe the relationship between the catheter 19C and medical element 13C.

In yet another alternative embodiment of the present invention, the basic catheter system can be equipped with a pacing lead for use as a coronary sinus catheter system. Turning now to the drawings, FIG. 1-D shows the coronary sinus catheter 11D in place in the human body. (The particular shape of the coronary sinus catheter in FIG. 1-D is illustrative only.) The catheter includes a pacing lead 13D adapted for placement in a vein on the left ventricle of a heart 15D having a right atrium 17D and a coronary sinus 19D for access to the vein disposed on the exterior of the left ventricle 21D. Typically, such systems also include an atrial lead 20D and a right ventricle lead 22D. The catheter itself has an outer member 23D having a distal end portion 25D capable of having a curve formed therein. It is preferred that the outer member be remotely controllable by a pull-wire or other conventional means to form the curve in the outer member remotely. As shown in FIG. 1-D, outer member 23D is sized to be disposed in at least one chamber of the mammalian heart 15D. The catheter 11D also includes an inner catheter 27D having a passage therein for containing at least a portion of the pacing lead 13D for placement thereof. The inner catheter has a distal end portion 29D capable of having a curve formed therein. As will become apparent in connection with the description below, the distal end portions of the outer member and the inner catheter are disposable with respect to each other so that they interact to allow cannulation of the coronary sinus 19D irrespective of the orientation of the coronary sinus with respect to the right atrium of the heart.

Although from FIG. 1-D one would conclude that cannulation of the coronary sinus is a straightforward process, that is not in fact the case. The orientations of the various parts of the heart shown in FIG. 1 are ideal and somewhat simplified. In practice, particularly with the diseased hearts for which the present invention is ideally designed, the placement and orientation of the coronary sinus is highly variable, and it is generally impossible to determine the exact shape that will be needed to cannulate the coronary sinus for placement of the pacing lead in the vein exterior of the left ventricle prior to actual insertion of the catheter and exploration. With conventional catheters, this process may require much experimentation and even the removal and replacement of the original catheter with a second (or even a third) catheter. All this exploration and replacement, of course, takes time and involves additional risk to the patient.

In FIG. 2-D catheter 11D is formed into an up-going shape. To form this shape, the outer member and inner catheter are moved translationally with respect to each other and the distal ends thereof are formed in curves to make the desired shape of the catheter 11D to cannulate the coronary sinus, and held in that shape for use of that shape in the procedure. As can be seen, the orientation of the coronary sinus in this case varies substantially from the idealized situation shown in FIG. 1-D.

In FIG. 3-D catheter 11D is formed into a down-going shape. Up-going and down-going in this application are defined by reference to the direction the outer member would move if it were moved further into the body. A curve pointing in the direction of further movement is up-going (FIG. 2-D), and a curve pointing opposite the direction of further movement is down-going (FIG. 3-D). In this shape, the catheter 11D may successfully cannulate the coronary sinus of FIG. 3-D which opens into the right atrium in a very different direction from that of FIGS. 1-D and 2-D.

In FIG. 4-D catheter 11D is formed into an out-of-plane shape. This is accomplished by curving the distal portion of outer member 25D to define a first plane P1 and rotating the curved distal end of inner catheter 27D so that it points along a second plane P2 which is not coinciding with plane P1. Out of plane shapes are particularly useful in cannulating a coronary sinus that lies in a plane substantially outside the plane of the outer member.

Turning to FIG. 5-D, catheter 11D is formed into a shape such that it is wedged against the wall of the right atrium. This is a particularly useful configuration, since it provides a solid foundation for the catheter as a whole during placement of the pacing lead adjacent the left ventricle.

It should be appreciated that catheter 11D is capable of forming all the shapes of FIGS. 2-D through 4-D (and infinite variations thereof) with the same two elements-outer member 25D and inner catheter 27D. By suitable translation and/or rotation of the outer member and inner catheter and adjustment of the curves in the distal end portions of each, an infinite number of shapes can be made, at least one of which may be used to successfully cannulate the coronary sinus. For example, FIG. 6-D shows a few of the infinite number of up-going shapes that catheter 11D may be formed into to successfully cannulate the coronary sinus. Similarly, FIGS. 7-D and 7A-D show a few of the infinite number of out-of-plane shapes that may be formed, and FIG. 8-D shows a few of the infinite number of down-going shapes that may be formed.

Catheter 11D is particular useful in connection with accessing vessels or orifices that are in unusual positions. As human bodies age, the relative positions of various vessels and orifices change, so that a catheter of a conventional shape to enter that vessel may not be shaped correctly to do so.

That is, the inner catheter and the outer member interact to form the desired shape, whether it be up-going, down-going, out-of-plane, or any desired combination thereof. It is preferred that both the inner and outer members be capable of being imaged (such as by fluoroscopy or direct visualization, for example), so that the desired shape may be easily and readily achieved.

In yet another alternative embodiment of the present invention, the basic catheter system can be used as an endovascular stent graft system. Turning now to the drawings, a method of the previous invention involves inserting a stent graft limb branch 11E into a stent graft body 13E disposed in a living being 15E. In FIG. 1-E, the limb branch 11E is shown in a collapsed state, and the portion of the living body shown is the aorta 16E in the abdomen below the level of the renal arteries. The aneurysm in the aorta is labeled 17E. The stent graft body 13E for ease of illustration is shown separated from the aorta walls, but it should be appreciated (and is known in the art) that the stent graft forms a tight seal in the conventional manner at both ends 13AE through 13EE and 13BE. The stent graft body 13E has a trunk 19E and an opening 21E for receiving branch 11E. For clarity (in FIG. 1A-E) limb branch 11E is shown in its expanded state in which it forms a unitary structure with the stent graft body 13E.

In FIG. 1-E a fairly ideal shape of the aorta and iliac arteries configuration is illustrated. In actual living beings, the configuration is often far from ideal. For example, in FIGS. 2A-E and 2B-E, aorta 15E is illustrated with the left and right iliac arteries 23E, 25E joining the aorta at various angles and with the iliac arteries themselves having various degrees of twists and turns. It should also be appreciated that the illustrations of FIGS. 1-E and 2-E are of necessity in two-dimensions, whereas the aorta and iliac arteries are three-dimensional structures with variation from ideal in all three dimensions. For example, in FIGS. 3A-E through 3C-E a few of the vast numbers of possible configurations of the iliac arteries with respect to the aorta are illustrated at the point. As illustrated in FIGS. 2A-E and 2B-E, the relative positions of the iliac arteries change considerably as the aorta is approached. The result of all this variation is that the catheter (guide wire, tube, etc.) carrying the limb branch of the stent graft may exit the iliac artery 25E at any number of different angles in three dimensions. This makes accessing opening 21E extremely difficult.

As shown in FIG. 4-E, the catheter 31E (shown in idealized form in FIG. 4-E) may miss opening 21 E in any of three different ways (or any combination thereof) as a direct result of the relative position of the iliac artery 25E with respect to the placement of the stent graft body. In the situation indicated by the left-most catheter 31AE emerging from iliac artery 25E (labeled 25AE to distinguish it from two other possible positions 25BE and 25CE), the catheter 31AE is disposed behind the body of stent graft 13E. This represents an error in attempted placement in the direction into and out of the plane of the paper (indicated by the plus/minus arrow 35E. Similarly, the middle catheter 31 BE emerging from the orientation illustrated by 25BE, misses the opening 21E to the left. Variation in this plane is from side-to-side in the plane of the paper (plus/minus arrow 37E). The rightmost illustration shows catheter 31BE missing the opening in the vertical direction (plus/minus arrow 39E). Of course, the actual misalignment in a given situation may be in all three dimensions, making insertion of the limb branch an extremely trying and time-consuming effort.

The present invention solves this problem by inserting a guide element (such as a guide wire or a guide catheter through a vessel of the living being to the vicinity of the branch receiving opening of the stent graft body. As illustrated in FIGS. 5A-E and 5B-E, the guide element may be two- or three-part. In FIG. 5A-E, the two-part guide element 41AE is shown to have an inner curved or curvable element 43E (such as a conventional guide wire) which interacts with an outer curved or curvable medical tube 45E. The inner element and outer tube are movable rotationally and translationally with respect to each other so that the curved or curvable distal end portions thereof may interact to form a vast multitude of possible shapes, including the exact shape needed to access opening 21E in the stent graft body 13E. In FIG. 5B-E, the three-part guide element 41BE is shown to have a straight inner element 47 (preferably a conventional guide wire) which is basically pointed in the correct direction by the interaction of a curved or curvable inner medical tube 49E and a curved or curvable outer medical tube 51E. Inner element 47E is movable translationally with respect to the inner medical tube 49E, and the inner and outer medical tubes 49E and 51E are movable both rotationally and translationally with respect to each other to form the vast variety of shapes described above. Once the necessary shape is formed, the inner element 47E is moved translationally with respect to the inner medical tube 49E to access the opening 21E in the stent graft body 13E.

In either case, the guide element 41E is thus manipulated in three dimensions to form a shape corresponding to the orientation of the branch receiving opening 21E with respect to the position of the vessel 25E, so that the guide element shape is varied, in situ, to the required shape despite any abnormalities in the shape and orientation of the vessel. Thereupon the guide element 41E, after formation into the proper shape, is inserted into the branch receiving opening 21E of the stent graft body 13E. Stent graft limb branch 11 is then fed over the guide element until the distal end of the limb branch is received in the branch receiving opening of the stent graft body.

Among the various shapes that can be achieved by the present method are the up-going shapes shown in FIGS. 5A-E and 5B-E, as well as down-going shapes (see FIG. 6-E) and out-of-plane shapes (see FIG. 7-E). An up-going shape is one in which the extreme distal end of the guide element points in the direction which corresponds to the direction of advancement of the catheter into the body. Similarly, a down-going shape is one in which the extreme distal end of the guide element points in a direction opposite to the direction of advancement of the catheter into the body. An out-of-plane shape is one which is out of the plane defined by the curved distal end portion of the outer medical tube. It should be understood that the particular up-going, down-going, and out-of-plane shapes are illustrative only, and that a vast number of such shapes may be generated as needed by the present invention. Of particular interest is the fact that the out-of-plane shapes (FIG. 7-E) may be either clockwise out-of-plane shapes (e.g., FIG. 7B-E) or counter-clockwise out-of-plane shapes (FIG. 7C-E) as the circumstances require. All these shapes are achieved by simply moving the curved/curvable elements translationally and rotationally with respect to each other. It should also be noted that these shapes are three-dimensional, so that (for example) up-going, out-of-plane or down-going, out-of-plane shapes may be made as well.

The guide element with its outer tube 45E or 51E also provides a stable platform for the feeding of the stent graft limb branch 11E. This can be done under either fluoroscopic or optical imaging. Although a particular stent graft and artery have been illustrated, the present invention is not limited in that way. For example, the stent graft body may be disposed in an abdominal artery such as a suprarenal abdominal artery or in a thoracic artery, or in any other artery in which stent grafts are placed.

Nor is the invention limited to the placement of stent grafts and stent graft limbs. The guide element disclosed herein is of general usefulness and may be used in those cases in which a conventional guide wire is typically used.

In yet another alternative embodiment of the present invention, the basic catheter system can be used as a nasogastric tube. Referring to the drawings, there is shown a nasogastric tube 11F being improperly inserted into the trachea T of a patient 13F. FIG. 2-F shows the proper placement in esophagus E. Note that the anatomical structures at the rear of the oral cavity along with the required curved shape to move from the nasal passages to either the trachea or the esophagus at times tend to naturally force a feeding tube or other nasogastric tube toward the trachea.

Similar problems may arise lower in the gastro-intestinal tract. For example, in FIGS. 3A-F and 3B-F an hiatus hernia 15F is disposed above the diaphragm D and stomach S. Tube 11F can (and in actual practice with conventional tubes does) curl around in the hernia and never reach the desired placement in the stomach. Although the solution to proper movement looks fairly straightforward in FIGS. 3A-F and 3B-F, it should be realized that the stomach and the hernia are not necessarily in the same plane. The hernia can be positioned dorsally or ventrally with respect to the stomach and the opening to the stomach can be at a wide variety of angles. Using conventional tubes, this is a serious problem.

Passing through the pyloric valve P into the duodenum is an equally exacting task, even in the best of conditions. Basically, any portion of the gastro-intestinal tract can provide difficult or unexpected situations which make placement of nasogastric tubes difficult. (See FIG. 3B-F, which illustrates the irregular, non-planar aspects of the problem.)

A nasogastric tube 11F of the present invention (FIGS. 4-F through 7-F) solves these problems. Tube 11F has a proximal end 17F (FIG. 1-F) and a distal end portion 19F. It includes an outer tube 23F sized to be inserted in a nasal passage of a living being 13F. The outer tube is sufficiently long to extend from the exterior of the living being through the nasal passage to stomach S. It is sufficiently flexible to adapt to the shape of passages through which it extends to said stomach. The outer tube 23F (like the entire nasogastric tube 11F) has a distal end portion, a proximal end, and a passage extending therethrough.

Tube 11F also includes an inner element 27F disposable in outer tube 23F and extending therethrough so that a distal end portion of the inner element is at least adjacent the distal end portion of the outer tube. The inner element is movable rotationally and translationally with respect to the outer tube so that the outer tube and inner element interact to vary the shape of the distal end portion of the nasogastric tube to facilitate the movement of the nasogastric tube from said nasal passage at least to stomach. Translational movement is indicated by the double-headed arrow in FIG. 5-F, while rotation is best shown in FIG. 6-F which shows the inner element rotated to two different positions with respect to outer tube 23F. It has been discovered that when the distal end portions of both the outer tube and the inner element are capable of assuming curved shapes a vast multitude of shapes of the composite nasogastric tube 11F can be formed (only a few of which are illustrated in FIGS. 4-F through 7-F). For example, in FIG. 4-F, bending outer tube 23F more or less (by a pull-wire or other conventional means) while bending and/or moving the inner element in and out translationally as indicated results in whole families of up-going shapes (an up-going shape being one pointing generally in the direction of insertion of the composite tube 11F).

In FIGS. 5-F and 6-F, with the outer tube bent in one plane and rotational movement of the inner curved element, there results whole families of out-of-plane shapes. In addition, in FIG. 7-F bending of the outer tube 23F and inner element 27F, along with translational movement if desired results in entire families of down-going shapes. (A down-going shape is one pointing generally opposite the direction of insertion of the composite tube 11F.) For purposes of this application, the distal ends of the outer tube and the inner element are those curved (or curvable) portions thereof which lie substantially in a plane. When the curves of the outer tube and the inner curved element are at 0 degrees or 180 degrees with respect to each other, the pure up-going and down-going shapes result, while in between those angles various out-of-plane shapes result (with pure out-of-plane shapes at 90 degrees and 270 degrees).

If nasogastric tube 11F is to be used as a feeding tube, it is necessary that either the outer tube 23F or the inner element 27F (or both) have a passage suitable for the passage of nutrients and an opening generally in the distal end portion to allow nutrients to be inserted into stomach S or beyond. In that case, tube 11F is provided with a suitable proximal port for removable attachment to a nutrient source.

It is also preferred, but not required, that nasogastric tube 11F include conventional fluoroscopic imaging markers 31F (see FIG. 4-F) in the area of the distal end portion of the tube to allow visualization of the shape of the distal end portion as the tube is inserted. Alternatively, the tube 11F can be radio-opaque to serve the same purpose.

Although the present invention is particularly well suited for avoiding many of the conventional problems in placing feeding tubes in the stomach, it should be appreciated that the shaping capabilities of this tube make it uniquely suited to solving the difficult problem of placing a tube through the pyloric valve P into the duodenum.

The foregoing description of the embodiments of the present invention has been provided for purposes of illustration and description. As various changes could be made in the above constructions without departing from the scope of the invention, it is intended that all matter contained in the above descriptions or shown in the accompanying drawings shall be interpreted as illustrative and not in a limiting sense.

It will also be seen in the above disclosure that several of the intended purposes of the invention are achieved, and other advantageous results obtained. Additionally, in the preceding description, numerous specific details are set forth such as examples of specific components, devices, methods, in order to provide a thorough understanding of embodiments of the present disclosure. It will be apparent to a person of ordinary skill in the art that these specific details need not be employed, and should not be construed to limit the scope of the disclosure.

The disclosure herein is also not intended to be exhaustive or to limit the invention to the precise forms disclosed. Individual elements or features of a particular embodiment are generally not limited to that particular embodiment, but, where applicable, are interchangeable and can be used in a selected embodiment, even if not specifically shown or described.

In the development of any actual implementation, numerous implementation-specific decisions must be made to achieve the developer's specific goals, such as compliance with system-related and business-related constraints. Such a development effort might be complex and time consuming, but is nevertheless a routine undertaking of design, fabrication and manufacture for those of ordinary skill. The scope of the invention should be determined by any appended claims and their legal equivalents, rather than by the examples given.

Terms such as “proximate,” “distal,” “upper,” “lower,” “inner,” “outer,” “inwardly,” “outwardly,” “exterior,” “interior,” and the like when used herein refer to positions of the respective elements as they are shown in the accompanying drawings, and the disclosure is not necessarily limited to such positions. Terms such as “first,” “second,” and other numerical terms when used herein do not imply a sequence or order unless clearly indicated by the context.

When introducing elements or features and the exemplary embodiments, the articles “a,” “an,” “the” and “said” are intended to mean that there are one or more of such elements or features. The terms “comprising,” “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It will be understood that when an element is referred to as being “connected,” “coupled,” “engaged,” or “engageable” to and/or with another element, it can be directly connected, coupled, engaged, engageable to and/or with the other element or intervening elements may be present. In contrast, when an element is referred to as being “directly connected,” “directly coupled,” “directly engaged,” or “directly engageable” to another element, there are no intervening elements present. Other words used to describe the relationship between elements should be interpreted in a like fashion (e.g., “between” versus “directly between,” “adjacent” versus “directly adjacent,” etc.).

What is claimed is:

1. A catheter system comprising:
a catheter tube having a wall and an internal lumen extending substantially through the length of the catheter tube, said catheter tube having an outside diameter sufficiently small so that the catheter tube may be inserted into the human body, said catheter tube having a proximal end and a distal end;
at least one wire running from the proximal end of the catheter tube to the vicinity of the distal end of the catheter tube, said wire being connected to the catheter tube adjacent the distal end so as to allow the distal tip of the catheter tube to be deflected upon movement of said wire;
said catheter tube having a substantially uniform transverse cross section throughout a substantial part of its length, the catheter tube wall adjacent the distal end of the catheter tube having therein on one side of the longitudinal axis of the catheter tube a predetermined region of weakness, whereby upon tension being applied to the wire the distal end of the catheter tube bends in the direction of the predetermined region of weakness;
an inner surgical element disposed in the lumen of the catheter tube wherein the inner surgical element is rotationally and axially movable in the lumen of the catheter tube and wherein the inner surgical element being sufficiently pliable to bend upon bending of the catheter tube, and wherein at least one of either the catheter tube or the inners surgical element is operatively connected to a medical tool; and
a controller device for manipulating at least one of either the catheter tube at a region near the distal end of the catheter tube, or the inner surgical element near the distal end of the inner surgical element, to configure the region near the distal end of the catheter system into a operational configuration that is one of either an up-going configuration, an down-going configuration, or an out of plane configuration, wherein the operational configuration is achieved without bracing any component of the catheter system against the patient's tissue, organ, or bone.

2. The catheter system of claim 1 wherein the predetermined region of weakness being formed by gaps in the wall of the catheter tube, at least some of the gaps having one of either a generally crescent moon shape, a generally diamond shape, or a generally arrowhead shape.

3. The catheter system of claim 1 further comprising at least one medical tool disposed near the distal end portion of at least one of either the catheter tube or the inner surgical element wherein the medical tool is used for medical treatment of the patient.

4. The catheter system of claim 3 wherein the medical tool is at least one of either an ablation tool or a mapping electrode including a receiver disposed at the distal end portion of at least one of either the catheter tube or the inner surgical element for one of either ablating a tissue or mapping signals respectively in the heart when the distal end portion of the electrophysiology catheter system is disposed in the heart.

5. The catheter system as set forth in claim 4 wherein the medical tool is an ablating tool disposed at the distal end portion of at least one of either the catheter tube and the inner surgical element for ablating a tissue in the organ when the distal end portion of the catheter system is disposed in the organ, wherein the ablating tool is selected from the group consisting of cryo-surgery ablation tools, laser ablation tools, microwave ablation tools, and radiofrequency ablation tools, and wherein the selected ablating tool is operatively connected to an ablation source.

6. The catheter system as set forth in claim 4 wherein the medical tool is a mapping electrode including a receiver disposed at the distal end portion of at least one of either the catheter tube or the inner surgical element for mapping signals in the heart.

7. The catheter system as set forth in claim 4 further comprising a pacing lead adapted for placement in a vein on the left ventricle of a heart having a right atrium and a coronary sinus wherein the catheter tube is sized to be disposed in at least one chamber of said mammalian heart, wherein the inner surgical element has a passage therein for containing at least a portion of the pacing lead for placement thereof, said inner surgical element having a curvable distal end portion, said distal end portions of the catheter tube and the inner surgical element are disposable with respect to each other so that the distal end portions interact to allow cannulation of the coronary sinus irrespective of the orientation of the coronary sinus with respect to the right atrium of the heart.

8. The catheter system as set forth in claim 4 further comprising:
a guide element sized to pass through a vessel of the living being to the vicinity of the branch receiving opening ready to receive a stent graft body, wherein said stent graft body having a trunk and an opening for receiving said branch, wherein said stent graft limb branch is movable over the guide element until the distal end of the limb branch is received in the branch receiving opening of the stent graft body;
means for manipulating the guide element in three dimensions to form a shape corresponding to the orientation of the branch receiving opening with respect to the position of the vessel, whereby the guide element shape is varied, in situ, to the required shape despite any abnormalities in the shape and orientation of the vessel.

9. The catheter system as set forth in claim 4 wherein the catheter tube is sized to be inserted in a nasal passage of a living being, wherein the catheter tube is sufficiently long to from at least the nasal passage to a stomach of the patient, wherein the catheter tube is sufficiently flexible to adapt to the shape of passages through which it extends to said stomach.

10. A method of directing and shaping an inner element by interaction with an outer catheter tube in which the inner element is disposed, said outer catheter tube having a diameter sufficiently small so that the catheter tube may be inserted into the human body, having a length substantially the length of the inner element, and having a bore therethrough running substantially from the proximal end of the catheter tube to the distal end of the catheter tube, said inner element being disposed in said bore and being axially movable with respect to the catheter tube in the bore, said catheter tube having at its distal end a longitudinally extending predetermined region of weakness, the distal end of the catheter tube being curvable in the direction of the predetermined region of weakness, said method comprising the steps of:
disposing the inner element at a first rotational position with respect to the predetermined region of weakness of the catheter tube such that the distal end of the inner element is disposed substantially in a plane containing the longitudinal axis of the predetermined region of weakness, and extending the inner element distally outwardly with respect to the catheter tube to generate the desired element shape;
disposing the inner element at a second rotational position with respect to the predetermined region of weakness of the catheter tube, said second rotational position being substantially 180 degrees from the first rotational position, and extending the inner element distally outwardly with respect to the catheter tube to generate the desired element shape; and
disposing the inner element at a third rotational position with respect to the predetermined region of weakness of the catheter tube, said third rotational position being disposed intermediate the first and second rotational positions, and extending the inner element distally outwardly with respect to the catheter tube to generate the desired element shape and.
deflecting the distal end of catheter tube such that the distal end of the inner element takes a desired shape.

11. A method of inserting a stent graft limb branch into a stent graft body disposed in a living being, said stent graft body having a trunk and an opening for receiving said branch, comprising the steps of:
inserting a catheter system element through a vessel of the living being to the vicinity of the branch receiving opening of the stent graft body wherein the catheter system comprises;
a catheter tube having a wall and an internal lumen extending substantially through the length of the catheter tube, said catheter tube having an outside diameter sufficiently small so that the catheter tube may be inserted into the human body, said catheter tube having a proximal end and a distal end;
at least one wire running from the proximal end of the catheter tube to the vicinity of the distal end of the catheter tube, said wire being connected to the catheter tube adjacent the distal end so as to allow the distal tip of the catheter tube to be deflected upon movement of said wire;
said catheter tube having a substantially uniform transverse cross section throughout a substantial part of its length, the catheter tube wall adjacent the distal end of the catheter tube having therein on one side of the longitudinal axis of the catheter tube a predetermined region of weakness, whereby upon tension being applied to the wire the distal end of the catheter tube bends in the direction of the predetermined region of weakness;
an inner surgical element disposed in the lumen of the catheter tube wherein the inner surgical element is rotationally and axially movable in the lumen of the catheter tube and wherein the inner surgical element being sufficiently pliable to bend upon bending of the catheter tube, and wherein at least one of either the catheter tube or the inner surgical element is operatively connected to a medical tool; and
a controller device for manipulating at least one of either the catheter tube at a region near the distal end of the catheter tube, or the inner surgical element near the distal end of the inner surgical element, to configure the region near the distal end of the catheter system into a operational configuration that is one of either an up-going configuration, an down-going configuration, or an out of plane configuration, wherein the operational configuration is achieved without bracing any component of the catheter system against the patient's tissue, organ, or bone;
manipulating the catheter system components in three dimensions to form a shape corresponding to the orientation of the branch receiving opening with respect to the position of the vessel, whereby the catheter system shape is varied, in situ, to the required shape despite any abnormalities in the shape and orientation of the vessel; inserting components of the catheter system into the branch receiving opening of the stent graft body; feeding the stent graft limb branch over the inner surgical element until the distal end of the limb branch is received in the branch receiving opening of the stent graft body.
